(19) United States
(12) Patent Application Publication
(10) Pub. No.: US 2025/0304959 A1
(43) Pub. Date: Oct. 2, 2025

(54) PROGRAMMABLE RECRUITMENT OF TRANSCRIPTION FACTORS TO ENDOGENOUS GENES

(71) Applicant: SRI INTERNATIONAL, Menlo Park, CA (US)

(72) Inventor: Kevin Luebke, Menlo Park, CA (US)

(21) Appl. No.: 18/865,482

(22) PCT Filed: Dec. 20, 2022
(86) PCT No.: PCT/US2022/082062

§ 371 Date: Nov. 13, 2024

(51) Int. Cl. C12N 15/113; A61K 48/00; C07K 14/47; C12N 7/00; C12N 9/22

(52) U.S. Cl. CPC C12N 15/113; A61K 48/005; C07K 14/4702; C12N 7/00; C12N 9/222; C07K 2319/09; C12N 2310/20; C12N 2710/10021; C12N 2740/15011; C12N 2750/14143

(57) ABSTRACT

The disclosure provides a method for modulating gene expression in a cell-specific manner in response to an intracellular or extracellular stimulus. The disclosure provides a platform entitled the PROTEGE platform, which comprises a DNA binding module and a transcription factor binding module, referred to herein as PGM. The PGM binds the promoter region of a target gene with sequence specificity through the DNA binding module and also binds a TF through the TF-binding module. When the TF is activated in response to an intracellular or extracellular stimulus, it binds the PGM and, due to its close proximity to the promoter of the gene, modulates expression of a target gene in response to the stimulus.

### CROSS-REFERENCE TO RELATED APPLICATIONS

This application claims the benefit of priority of U.S. Provisional Application No. 63/341,820, filed on May 13, 2022, the contents of which are incorporated herein by reference in their entirety.

### FIELD

This disclosure is in the field of programmable modulation of gene expression in a cell-specific manner by recruitment of transcription factors to one or more genes in response to intracellular and/or extracellular stimuli. The disclosure provides a platform designated herewith as the Protege Platform.

### REFERENCE TO SEQUENCE LISTING SUBMITTED ELECTRONICALLY

The instant application contains a Sequence Listing which has been submitted electronically in XML format and is hereby incorporated by reference in its entirety. Said .XML copy is 73,779 kilobytes in size.

### BACKGROUND

Organisms respond to disease and injury by modulating their expression of specific genes to promote recovery, healing, or disease resistance. Cells sense external signals arising from disease or injury and respond by activating transcription factors that modulate the expression of genes under their control. However, genes that could benefit healing, recovery, or disease resistance are often not regulated to realize their beneficial effects. This deficiency can be due to the gene not being under the control of relevant transcription factors or due to insufficient activation or repression of the gene by the relevant transcription factors.

A conventional solution to this problem is to administer the product encoded by the potentially beneficial gene as a pharmaceutical agent. For example, recombinant human bone morphogenetic protein-2 (rh-BMP-2), is used to promote recovery after spinal surgery. Similarly, recombinant human platelet-derived growth factor (rhPDGF) is applied to diabetic ulcers to improve wound healing. This approach is often unsuccessful or of limited usefulness because it does not restrict the activity of the added gene product to the time and place where it is needed, resulting in compensatory effects or negative side-effects.

Another approach, made possible by recent advances in gene editing, is to modify the genome to alter the expression of therapeutic gene products. For example, mutations or polymorphisms that lead to a deficiency of a particular gene product can be altered to establish beneficial gene expression levels. Such modification uses gene editors comprising a sequence-specific DNA binding component and an endonuclease that cleaves the DNA at or near the site of binding. Alterations at the site of cleavage can be made by homology directed repair (HDR), in which exogenous DNA containing the desired edited sequence acts as the repair template.

Several types of sequence-specific nucleases have been used for gene editing, including zinc finger nucleases (ZFNs), transcription activator-like effector nucleases (TALENs), and CRISPR endonucleases. For example, a ribonucleoprotein complex comprising the Cas9 (CRISPR-associated protein 9) endonuclease and a guide RNA can bind to and cleave DNA genomic sequences specified by the guide RNA. Gene editing carries the risks of off-target editing and that the edits made are permanent. Thus, deleterious off-target edits or intended edits found to have deleterious effects are irreversible.

Gene expression can be modulated reversibly with synthetic transcription factors. Like naturally occurring transcription factors, synthetic transcription factors bind to specific sequences in the promoter or enhancer regions of genes and deliver or recruit endogenous factors to promote or interfere with assembly of the transcription initiation complex or promote chromatin modifications that modulate transcription. Synthetic transcription factors have been created using zinc fingers, TALEs, and CRISPR-associated (Cas) proteins modified to eliminate their endonuclease activity. For example, Dead Cas9 (dCas9), is a mutated form of Cas9 whose endonuclease activity has been disabled through mutations in its endonuclease domains. It remains capable of binding to its guide RNA and the targeted DNA strand. Transcription factors linked to dCas9 or its bound guide RNA can be delivered to target DNA sequences in the promoter or enhancer regions of genes and modulate their transcription. Transcription factors that have been used in this context include Vp64, p65, Hsf1, and the Epstein-Barr virus R transactivator (Rta). The transcription factors that have been used previously for this purpose have been non-native to the treated cell (e.g., viral transcription factors in mammalian cells) and/or artificially and covalently fused to the dCas9 or other proteins that mediate their binding to dCas9. Therefore, they have not been endogenously produced transcription factors for which activity is dependent on physiological signals that affect the cell.

Significant risk of irreversible, harmful genetic modification may occur from nucleic acid therapies (e.g., ASO, antagomirs, siRNA, therapeutic mRNA) to modulate gene expression. Furthermore, the action of these therapies is not limited to the physiological conditions under which they are needed. Except for mRNA, the capacity of these approaches to increase expression of a beneficial gene is limited.

### SUMMARY

Cells respond to disease and injury by expressing genes in response to environmental cues that call for them. But not all the genes that could promote healing and recovery are expressed at the optimal level or at all. Current medical measures aimed at artificially providing the products of beneficial genes are often ineffective or harmful because they do not limit their action to the place in the body and time that they are needed. What is needed is a way to turn genes on (or off) in response to physiological signals that indicate a benefit for the modulation of the gene.

The activities of artificial transcription factors reported previously do not respond to environmental signals that arise from disease or injury. This response can be attained by directing the activities of endogenous transcription factors that are activated by environmental signals associated with disease or injury to the target genes. Reversibly modulating the expression levels of targeted genes in a manner such that the expression levels of those genes depend on environmental signals arising from disease or injury will limit the alteration of gene expression levels to the cells, cellular locations, and times when the altered gene expression will have therapeutic effect. As such, it will avoid alteration of gene expression in cells, cellular locations, and times in which altered gene expression will have negative side effects.

Previously described modulators of gene expression have not directly incorporated native, endogenously produced transcription factors in their designs. For example, previous designs based on dCas9 have linked the transcription modulation domains to the dCas9-guide RNA ribonucleoprotein complex by direct fusion to the dCas9 protein, by fusion with a bacteriophage coat protein (MS2) that binds to an RNA sequence incorporated into the guide RNA, or by conjugation to antibodies that bind to polypeptide sequences fused to the dCas9 protein. The need for the transcription modulation domain to be covalently conjugated to another protein (e.g., dCas9, MS2, or antibody) in each of these approaches necessitates delivering the transcription modulation domain exogenously or by transfection with an expression vector for the fusion protein. This requirement prevents the direct delivery or recruitment of endogenous transcription factors to genes of interest by CRISPR-based DNA binding agents.

We describe herein compositions and methods for the transcriptional modulation of specific genes of interest using artificial transcription factors that simultaneously bind to specific sequences of genomic DNA within or proximal to target genes and one or more endogenously produced, native transcription factors that are activated in response to external signals. The principle is shown schematically in FIG. 1. The target gene is programmed by the sequence of the crRNA component of the guide RNA and the transcription factor(s) to which transcriptional modulation responds is (are) programmed by the transcription factor response element(s) incorporated into the guide nucleic acid. The following embodiments are non-limiting examples of the inventions provided in the disclosure:
1. An engineered non-naturally occurring system comprising a programmable gene modulator (PGM) for reversibly modifying expression of a target gene of interest in a cell in response to one or more intracellular or extracellular environmental signal(s), or the sgCNA subcomponent thereof, comprising the following subcomponents:
an endonuclease-defective DNA-binding polypeptide (preferably, a dCas polypeptide); and
a chimeric nucleic acid (sgCNA) comprising a CRISPR RNA (crRNA), a trans-activating crRNA (tracrRNA), and at least one nucleic acid segment comprising at least one transcription factor binding site;
wherein the crRNA comprises a sequence complementary to a nucleic acid sequence in the promoter region of the target gene of interest and each transcription factor binding site(s) in the PGM bind(s) to at least one endogenous transcription factor that is activated in a cell comprising the PGM in response to the environmental signal(s) and then recognizes and binds to the transcription factor binding site of the PGM which is bound through the crRNA to the promoter of the gene of interest, thereby bringing the transcription factor into proximity with the gene of interest and activating or suppressing expression of the gene of interest in response to the environmental signal(s).
2. The engineered non-naturally occurring system of embodiment 1, or the sgCNA subcomponent thereof, wherein (i) at least one transcription factor binding site in the PGM is also present in the target gene and/or (ii) at least one transcription factor binding site in the PGM is not an endogenous transcription factor binding site in the target gene.
3. The engineered non-naturally occurring system of any one of embodiments 1 and 2, or the sgCNA subcomponent thereof, wherein the PGM recruits the endogenous transcription factor(s) to the gene of interest when the endogenous transcription factor(s) has/have been activated in response to an environmental signal(s), thereby activating gene expression in response to the environmental signal(s) in a cell-specific manner.
4. The engineered non-naturally occurring system of any one of embodiments 1 to 3, or the sgCNA subcomponent thereof, wherein the transcription factor(s) is/are identified as a transcription factor that is (i) known to be activated in response to the environmental signal and (ii) known or not known to activate/inhibit expression of the target gene of interest.
5. The engineered non-naturally occurring system of any one of embodiments 1 to 4, or the sgCNA subcomponent thereof, wherein the DNA-binding polypeptide is a nuclease-deficient cas polypeptide.
6. The engineered non-naturally occurring system of any one of embodiments 1 to 5, or the sgCNA subcomponent thereof, wherein the gene of interest is identified as a gene whose expression (a) produces a beneficial cellular response to the environmental signal(s) but whose expression is undetectable or is increased by the PGM relative to the gene expression level in the absence of the PGM(s); or (ii) produces a detrimental effect to the cell and its expression is decreased by the PGM in response to the environmental signal(s), relative to the gene expression level in the absence of the PGM(s).
7. The engineered non-naturally occurring system of any one of embodiments 1 to 6, or the sgCNA subcomponent thereof, wherein the gene of interest encodes a protein, a microRNA, or a long noncoding RNA.
8. The engineered non-naturally occurring system of any one of embodiments 1 to 7, or the sgCNA subcomponent thereof, wherein the signal is any physical signal such as a light signal (e.g., UV light), ionizing radiation, heat/temperature, hyperosmotic or hypoosmotic conditions; a mechanical signal such as pressure (e.g., touch), movement of sound waves, and/or blood pressure; and/or any chemical signal such as a growth factor, a cytokine, a chemokine, cyclic AMP, a hormone, a neurotransmitter, an extracellular matrix component, a bacterial antigen, a viral antigen, a lipid, a lipopolysaccharide, gas levels (e.g, oxygen levels, nitric oxide levels), ion levels (e.g., calcium levels, sodium levels), pH, a reactive oxygen species, a heavy metal, oxidized LDL, and/or free radical, a cell-cell signal (e.g., T-cell binding, cell-cell contact), or a combination thereof.
9. The engineered non-naturally occurring system of any one of embodiments 1 to 8, or the sgCNA subcomponent thereof, wherein the transcription factor is selected from forkhead transcription factors, nuclear receptors, POU-domain proteins, SMAD, preferably Nrf2, FOX01, NF-kB, USF2, NFAT, EGR1, STAT3, and/or SREBP.
10. The engineered non-naturally occurring system of any one of embodiments 1 to 9, or the sgCNA subcomponent thereof, or the sgCNA subcomponent thereof, wherein the TF-binding module comprises (i) at least one TF-Binding segment (TFBS), wherein the TF-binding segment comprises DNA and/or RNA or (ii) wherein the TF-binding module comprises at least one TF-Binding segment (TFBS), wherein the TF-binding segment comprises a DNA aptamer or RNA aptamer selected for binding to the endogenous transcription factor.
11. The engineered non-naturally occurring system of any one of embodiments 1 to 10, or the sgCNA subcomponent thereof, wherein the TF-binding module comprises a sequence derived from a naturally occurring RNA.
12. The engineered non-naturally occurring system of any one of embodiments 1 to 11, or the sgCNA subcomponent thereof, wherein the TF-binding segment (TFBS) comprises a double-stranded segment of DNA containing at least one TF response element.
13. The engineered non-naturally occurring system of any one of embodiments 1 to 12, or the sgCNA subcomponent thereof, wherein the strands of the DNA portion of the sgRNA form a duplex and are joined by a loop sequence of any length.
14. The engineered non-naturally occurring system of embodiment 13, or the sgCNA subcomponent thereof, in which the loop comprises four nucleotides.
15. The engineered non-naturally occurring system of any one of embodiments 13 and 14, or the sgCNA subcomponent thereof, wherein the sequence of the loop comprises 5′-guanosine-adenosine-adenosine-adenosine-3′.
16. The engineered non-naturally occurring system of any one of embodiments 1 to 15, or the sgCNA subcomponent thereof, wherein the crRNA comprises at least 10, 11, 12, 13, 14, 15, 16, 17, 18, 19, 20, 21, 22, 23, 24, or 25 contiguous nucleobases that are at least 80%, 85%, 90%, 95%, 98%, 99%, or 100% complementary to the target nucleic acid sequence of the target gene of interest.
17. The engineered non-naturally occurring system of any one of embodiments 1 to 16, or the sgCNA subcomponent thereof, wherein the endonuclease-defective sequence-specific DNA binding protein (preferably, a dCas polypeptide) is fused with at least one copy of a nuclear localization signal.
18. The engineered non-naturally occurring system of any one of embodiments 1 to 17, or the sgCNA subcomponent thereof, wherein the crRNA comprises any one of the following sequences: SEQ ID NO:6 to SEQ ID NO:34.
19. The engineered non-naturally occurring system of any one of embodiments 1 to 18, or the sgCNA subcomponent thereof, wherein the transcription factor binding sequence comprises one or more sequences selected from SEQ ID NO: 1, 2, 3, 5, and those of Table 2.
20. The engineered non-naturally occurring system of any one of embodiments 1 to 19, or the sgCNA subcomponent thereof, wherein the tracrRNA binds dCas9.
21. The engineered non-naturally occurring system of any one of embodiments 1 to 20, or the sgCNA subcomponent thereof, wherein the crRNA, TFBS, and tracrRNA of the PGM are assembled in any one of the following molecular arrangements:
5′-crRNA-TFBS-tracrRNA-3′;
5′-crRNA-tracrRNA′-TFBS-tracrRNA″-3′ (wherein the TFBS is integrated anywhere within the sequence of the tracrRNA, including as an extension to one or more of its hairpin structures);
5′-crRNA-tracrRNA-TFBS-3′;
5′-crRNA-TFBS-tracrRNA′-TFBS-tracrRNA″-3′;
5′-crRNA-TFBS-tracrRNA-TFBS-3′;
5′-crRNA-tracrRNA′-TFBS-tracrRNA″-TFBS-3′;
5′-crRNA-TFBS-tracrRNA′-TFBS-tracrRNA″-TFBS-3′;
5′-crRNA-TFBS-tracrRNA′-TFBS-tracrRNA″-TFBS-tracrRNA′″-3′;
5′-crRNA-TFBS-tracrRNA′-TFBS-tracrRNA″-TFBS-tracrRNA′″-TFBS-tracrRNA″″-3′;
5′-crRNA-tracrRNA′-TFBS-tracrRNA″-TFBS-tracrRNA′″-TFBS-3′;
5′-crRNA-tracrRNA′-TFBS-tracrRNA″-TFBS-tracrRNA′″-TFBS-tracrRNA″″-TFBS-3′;
5′-crRNA-TFBS-tracrRNA-TFBS-tracrRNA-TFBS-tracrRNA-TFBS-3′;
5′-crRNA-TFBS-tracrRNA-TFBS-tracrRNA-TFBS-tracrRNA-TFBS-tracrRNA-TFBS-3′;
5′-crRNA-tracrRNA′-TFBS-tracrRNA″-TFBS-tracrRNA′″-3′;
5′-crRNA-tracrRNA′-TFBS-tracrRNA″-TFBS-tracrRNA′″-TFBS-tracrRNA″″-3′;
Wherein tracrRNA′, tracrRNA″, tracrRNA′″, and tracrRNA′″ are successive segments of the complete tracrRNA sequence.
22. The engineered non-naturally occurring system of any one of embodiments 1 to 21, or the sgCNA subcomponent thereof, wherein the PGM comprises one or more different TFBS, including response elements to multiple different transcription factors.
23. The engineered non-naturally occurring system of any one of embodiments 1 to 22, or the sgCNA subcomponent thereof, wherein the PGM comprises a nucleic acid backbone with one or more different TFBS wherein continuity of the nucleic acid backbone is broken at one or more positions and the full nucleic acid sequence assembles by base pairing of nucleotides from different strands.
24. The engineered non-naturally occurring system of embodiment 23, or the sgCNA subcomponent thereof, wherein the discontinuity of the nucleic acid backbone is within one or more TFBS.
25. The engineered non-naturally occurring system of any one of embodiments 1 to 24, or the sgCNA subcomponent thereof, wherein the TFBS is separated from the crRNA or tracrRNA by a linker of at least 1, 5, 10 20, or 30 DNA, RNA, or modified nucleotides.
26. An isolated nucleic acid comprising any one or more of the sgCNA, crRNA, tracrRNA, transcription factor binding site, or any other segment of the sgCNA of the PGM of any one of embodiments 1 through 25; or encoding the DNA binding protein of the PGM of any one of embodiments 1 through 25.
27. The isolated nucleic acid of embodiment 26, wherein the nucleic acid contains one or more modified or non-natural nucleotides.
28. The isolated nucleic acid of any one of embodiments 26 and 27, wherein the nucleic acid is 5, 10, 11, 12, 13, 14, 15, 16, 17, 18, 19, 20, 21, 22, 23, 24, 25, 26, 27, 28, 29, 30, 35, 40, 45, 50, 100, 200, 300, 400, 500, 1-5, 5-10, 10-20, 20-30, 30-40, 40-50, 50-60 60-70, 70-80, 80-90, 90-100, 100-125, 125-150, 150-200, 200-300, 300-400, or 400-500 bases long.
29. A vector comprising an isolated nucleic acid of any one of embodiments 26 to 28 under the control of a heterologous promoter, preferably wherein the vector is an AAV vector or another vector.
30. A virus comprising an isolated nucleic acid of any one of embodiments 26 to 28, preferably wherein the virus is a lentivirus or adenovirus.
31. A cell comprising a PGM, or sgCNA subcomponent thereof, of any one of embodiments 1 through 25, and/or a nucleic acid of any one of embodiments 26 through 28, and/or a vector of embodiment 29, and/or a virus of embodiment 30.
32. The cell of embodiment 31, wherein the cell is a prokaryotic cell or eukaryotic cell, preferably a mammalian cell, a cell of a non-human primate, or a human cell.
33. A composition comprising a PGM, or sgCNA subcomponent thereof, of any one of embodiments 1 through 25, a nucleic acid of any one of embodiments 26 through 28, a vector of embodiment 29, a virus of embodiment 30, a cell of embodiment 31, or a combination thereof.
34. The composition of embodiment 33, further comprising a cationic or ionizable lipid or cationic or ionizable polymer, preferably in a nanoparticle.
35. The composition of any one of embodiments 33 and 34, wherein the composition is a pharmaceutical composition further comprising a pharmaceutically acceptable excipient.
36. A method for reversibly modifying expression of a target gene of interest in a cell in response to one or more intracellular or extracellular environmental signal(s), comprising contacting the cell with the PGM, or the sgCNA subcomponent thereof, of any one of embodiments 1 through 25, a nucleic acid of any one of embodiments 26 through 28, a vector of embodiment 29, a virus of embodiment 30, a composition of any one of embodiments 33 through 35, or a combination thereof.
37. The method of embodiment 36, wherein the cell is a prokaryotic cell or eukaryotic cell, preferably a mammalian cell, a cell of a non-human primate, or a human cell.
38. A method of treating a disease, disorder, or injury in a subject in need thereof, the method comprising administering to the subject a therapeutically effective amount of the PGM, or the sgCNA subcomponent thereof, of any one of embodiments 1 through 25, an isolated nucleic acid of any one of embodiments 26 through 28, a vector of embodiment 29, a virus of embodiment 30, a cell of any one of embodiments 31 and 32, a composition of any one of embodiments 33 through 35, or a combination thereof.
39. The method of embodiment 38, wherein the disease, disorder, or injury is selected from cellular stress, an excisional or incisional wound, radiation exposure, viral or bacterial infection, sepsis, diabetic nephropathy, atherosclerosis, cystic fibrosis, Alzheimer's disease, oxidative stress, ischemia-reperfusion injury, inflammation, cancer, anti-cancer agent resistance, a genetic disease, or any other proliferative disease or disorder, inflammatory disease or disorder, autoimmune disease or disorder, liver disease or disorder, spleen disease or disorder, lung disease or disorder, hematological disease or disorder, neurological disease or disorder, gastrointestinal (GI) tract disease or disorder, genitourinary disease or disorder, infectious disease or disorder, musculoskeletal disease or disorder, endocrine disease or disorder, metabolic disease or disorder, immune disease or disorder, central nervous system (CNS) disease or disorder, neurological disease or disorder, ophthalmic disease or disorder, or a cardiovascular disease or disorder.
40. The method of embodiment 38, wherein the disease, disorder, or injury is selected from an excisional or incisional wound, radiation exposure, viral or bacterial infection, sepsis, diabetic nephropathy, atherosclerosis, cystic fibrosis, Alzheimer's disease, oxidative stress, ischemia-reperfusion injury, inflammation, and cancer.
41. A kit comprising the PGM, or the sgCNA subcomponent thereof, of any one of embodiments 1 through 25, a nucleic acid of any one of embodiments 26 through 28, a vector of embodiment 29, a virus of embodiment 30, a composition of any one of embodiments 33 through 35, or a combination thereof, and a container and/or instructions for using the kit.

### BRIEF DESCRIPTION OF THE DRAWINGS

The patent or application file contains at least one drawing executed in color. Copies of this patent or patent application publication with color drawings will be provided by the U.S. Patent and Trademark Office upon request and payment of the necessary fee.

FIG. 1A: Principle for a physiologically responsive gene expression modulator. A transcription factor (TF) is activated by a physiologic stimulus. Examples of physiologic stimuli include, but are not limited to, oxidative stress or growth factor signaling. The responsive gene expression modulator is a ribonucleoprotein complex composed of a disabled CRISPR-associated protein, such as dCas9, and a chimeric guide nucleic acid. The chimeric guide nucleic acid comprises a DNA hairpin that incorporates a binding site for the activated TF, a crRNA sequence, and a tracrRNA sequence. This complex binds to a sequence of genomic DNA proximal to a target gene that is to be made responsive to the physiologic signal. The binding site is programmed by the crRNA sequence in the guide nucleic acid. Association of the activated TF with the bound dCas9 complex brings the TF in proximity to the target gene resulting in modulation of target gene transcription. FIG. 1B: schematic of PGM in action FIG. 1C: Description of the crRNA module, tracrRNA module, and transcription factor binding site module.

FIG. 2A: Schematic of a conventional structure of a sgRNA, while FIG. 2B show exemplary embodiment of a chimeric guide nucleic acid (sgCNA) synthesis noting modules included according to the disclosure.

FIG. 3 provides various non-limiting examples of different applications of the PROTEGE Platform.

FIG. 4A exemplifies the need for a means to turn on therapeutic genes at the right time and place. FIG. 4B. exemplifies how the PROTEGE Platform enables controlled, reversible expression of therapeutic genes in response to injury or disease without altering genomic content.

FIGS. 5A-5D: Demonstration of a physiologically responsive programmable gene modulator (PGM) recruiting an activated transcription factor to a target DNA sequence. 5A. Experimental design. The target DNA sequence is a 20-base pair sequence (pink and gold) contained within a DNA duplex immobilized in the well of a multi-well plate. The gene modulator comprises dCas9 (yellow circle) complexed with a single guide nucleic acid comprising a crRNA module (turquoise) complementary to the target sequence, a tracrRNA module (teal), and a DNA module that forms a hairpin structure incorporating the Nrf2 response element in its stem (red). Binding of the gene modulator to the immobilized target DNA is followed by addition of a nuclear extract from HEK293 cells that have been treated with tert-butylhydroquinone (tBHQ) to stimulate activation and nuclear localization of Nrf2. After washing the well to remove unbound Nrf2, bound Nrf2 is detected with an anti-Nrf2 antibody, visualized by optical absorbance at 450 nm after treatment with HRP-conjugated anti-rabbit secondary antibody and development with HRP substrate. In FIGS. 5B-5D, each value is the mean of three replicates in separate wells. Error bars are the standard deviation in the mean. FIG. 5B. Dependence of Nrf2 binding on presence of the PGM. For “-PGM” wells, PBS was added instead of PGM solution. FIG. 5C. Dependence of Nrf2 binding on presence of target DNA sequence immobilized in well. In the “-Target DNA Sequence” wells, the immobilized duplex contained a scrambled version (same sequence composition, different sequence) of the target sequence in place of the target sequence. FIG. 5D. Dependence of Nrf2 binding on Nrf2 activation. Nuclear extract added to “-Nrf2” wells was from cells untreated with tBHQ.

FIGS. 6A and 6B: Nrf2-dependent modulation of klotho transcription in cultured cells. FIG. 6A. Human embryonic kidney cells were treated with a PGM targeted to the klotho promoter and containing the Nrf2 response element. After 16 hours, Nrf2 was activated with tBHQ. Total RNA was isolated after an additional 24 hours, and klotho expression relative to GAPDH expression was measured by RT-qPCR. FIG. 6B. Relative expression of klotho normalized to expression without addition of PGM or tBHQ. Values are means of three biological replicates and error bars are the standard deviations. P values are calculated from one-way ANOVA.

### DETAILED DESCRIPTION

In order for the present disclosure to be more readily understood, certain terms are first defined below. Additional definitions for the following terms and other terms are set forth throughout the Specification.

As used in this Specification and the appended claims, the singular forms “a,” “an” and “the” include plural referents unless the context clearly dictates otherwise.

Unless specifically stated or obvious from context, as used herein, the term “or” is understood to be inclusive and covers both “or” and “and”.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.,” as used herein, are used merely by way of example, without limitation intended, and should not be construed as referring only those items explicitly enumerated in the specification.

The terms “or more,” “at least,” “more than,” and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality,” “at least two,” “two or more,” “at least second,” and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000, or more. Also included is any greater number or fraction in between.

Throughout the specification the word “comprising,” or variations such as “comprises,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., +10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

Unless defined otherwise, all technical and scientific terms used herein have the same meaning as commonly understood by one of ordinary skill in the art to which this disclosure is related. For example, Juo, “The Concise Dictionary of Biomedicine and Molecular Biology”, 2nd ed., (2001), CRC Press; “The Dictionary of Cell & Molecular Biology”, 5th ed., (2013), Academic Press; and “The Oxford Dictionary Of Biochemistry And Molecular Biology”, Cammack et al. eds., 2nd ed, (2006), Oxford University Press, provide those of skill in the art with a general dictionary for many of the terms used in this disclosure.

The terms “transduction” and “transduced” refer to the process whereby foreign DNA is introduced into a cell via viral vector (see Jones et al., “Genetics: principles and analysis,” Boston: Jones & Bartlett Publ. (1998)). In some embodiments, the vector is a retroviral vector, a DNA vector, a RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof.

A “therapeutically effective amount,” “effective dose,” “effective amount,” or “therapeutically effective dosage” of a therapeutic agent, e.g., PGM, small molecules, “agents” described in the specification, is any amount that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. Such terms may be used interchangeably. The ability of a therapeutic agent to promote disease regression may be evaluated using a variety of methods known to the skilled practitioner, such as in human subjects during clinical trials, in animal model systems predictive of efficacy in humans, or by assaying the activity of the agent in in vitro assays. Therapeutically effective amounts and dosage regimens can be determined empirically by testing in known in vitro or in vivo (e.g., animal model) systems.

The term “combination” refers to either a fixed combination in one dosage unit form, or a combined administration where a compound of the present invention and a combination partner (e.g., another drug as explained below, also referred to as “therapeutic agent” or “agent”) may be administered independently at the same time or separately within time intervals, especially where these time intervals allow that the combination partners show a cooperative, e.g., synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time.

The term “genetically engineered” or “engineered” refers to a method of modifying the genome of a cell, including, but not limited to, deleting a coding or non-coding region or a portion thereof or inserting a coding region or a portion thereof.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95%, or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, or more bases in length such that the region of homology has sufficient homology to undergo binding with the corresponding genomic region. When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide, or portion thereof. In some embodiments, the homology or identity is assessed over a functional portion or a specified portion of the length. Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm, which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). A skilled person understands that amino acid (or nucleotide) positions may be determined in homologous sequences based on alignment.

A “patient” or a “subject” as used herein includes any human who is afflicted with a disease or disorder. The terms “subject” and “patient” are used interchangeably herein. A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey) or mouse). The term “patient” refers to a subject in need of treatment of a disease, disorder, or injury. In some embodiments, the subject is human. In some embodiments, the patient is human. The human may be a male or female at any stage of development. A subject or patient “in need” of treatment of a disease, disorder, or injury includes, without limitation, those who exhibit any risk factors or symptoms of a disease, disorder, or injury. In some embodiments, a subject is a non-human experimental animal (e.g., a mouse, rat, dog, or pig)

As used herein, the term “in vitro cell” refers to any cell which is cultured ex vivo. In particular, an in vitro cell may be an eukaryotic cell or a prokaryotic cell. The term “in vivo” means within the patient.

The terms “peptide,” “polypeptide,” and “protein” are used interchangeably, and refer to a compound comprised of amino acid residues covalently linked by peptide bonds. A protein or peptide contains at least two amino acids, and no limitation is placed on the maximum number of amino acids that may comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, a “tissue” is a group of cells and their extracellular matrix from the same origin. Together, the cells carry out a specific function. The association of multiple tissue types together forms an organ. The cells may be of different cell types. In some embodiments, a tissue is an epithelial tissue. Epithelial tissues are formed by cells that cover an organ surface (e.g., the surface of the skin, airways, soft organs, reproductive tract, and inner lining of the digestive tract). Epithelial tissues perform protective functions and are also involved in secretion, excretion, and absorption. Examples of epithelial tissues include, but are not limited to, simple squamous epithelium, stratified squamous epithelium, simple cuboidal epithelium, transitional epithelium, pseudostratified epithelium, columnar epithelium, and glandular epithelium. In some embodiments, a tissue is a connective tissue. Connective tissues are fibrous tissues made up of cells separated by non-living material (e.g., an extracellular matrix). Connective tissues provide shape to organs and hold organs in place. Connective tissues include fibrous connective tissue, skeletal connective tissue, and fluid connective tissue. Examples of connective tissues include, but are not limited to, blood, bone, tendon, ligament, adipose, and areolar tissues. In some embodiments, a tissue is a muscular tissue. Muscular tissue is an active contractile tissue formed from muscle cells. Muscle tissue functions to produce force and cause motion. Muscle tissue includes smooth muscle (e.g., as found in the inner linings of organs), skeletal muscle (e.g., as typically attached to bones), and cardiac muscle (e.g., as found in the heart, where it contracts to pump blood throughout an organism). In some embodiments, a tissue is a nervous tissue. Nervous tissue includes cells comprising the central nervous system and peripheral nervous system. Nervous tissue forms the brain, spinal cord, cranial nerves, and spinal nerves (e.g., motor neurons). In certain embodiments, a tissue is brain tissue. In certain embodiments, a tissue is placental tissue. In some embodiments, a tissue is heart tissue.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed (e.g., prophylactically (as may be further described herein) or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after using the methods disclosed herein and observing an alteration in spatiotemporal gene expression of one or more nucleic acids of interest in a cell or tissue in comparison to a healthy cell or tissue, or tissue not modified by the methods disclosed herein. The term “treatment” may also refer to the return of a cell to a physiological state, and encompasses reversal of cellular stress, prevention of cell death, return to normal growth, and the like.

The terms “tumor,” “cancer,” and “neoplasm” are used herein refers to an abnormal mass of tissue wherein the growth of the mass surpasses and is not coordinated with the growth of a normal tissue. A tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

### The Protege Platform: Programmable Gene Modulators

In one embodiment, the disclosure provides a platform for rational generation of therapeutic measures that harness beneficial genes to respond to disease or injury, only in the cells requiring the therapeutic response. In one embodiment, the platform uses a molecular device, a Programmable Gene Modulator (“PGM”), to recruit transcription factors that respond to a physiological condition of disease or injury to therapeutic genes of choice. A PGM for a given therapeutic goal may be designed from base pairing rules, known transcription factor binding DNA sequences, and known genomic sequences.

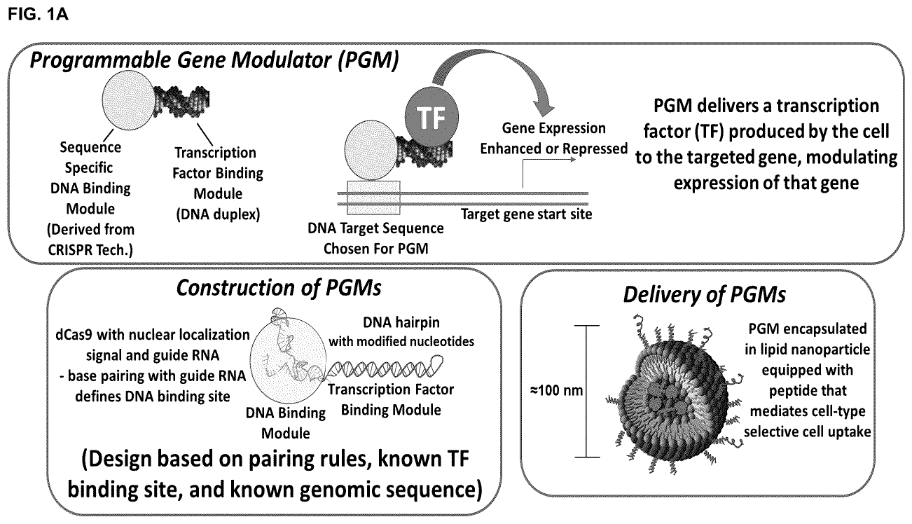

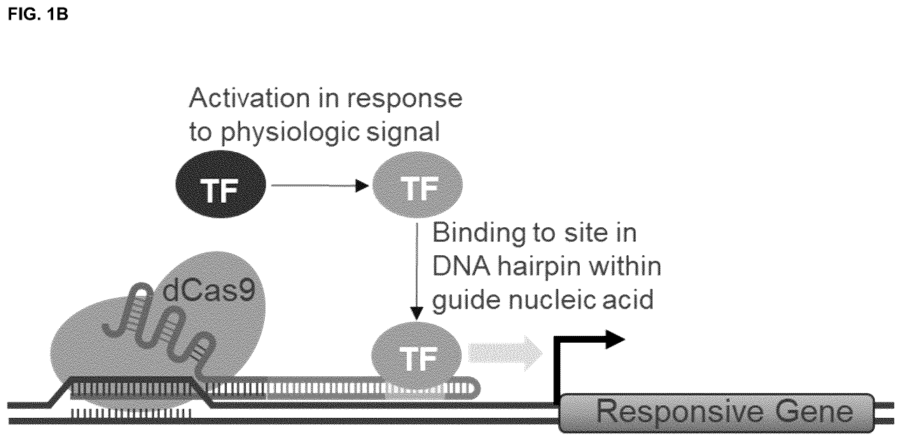

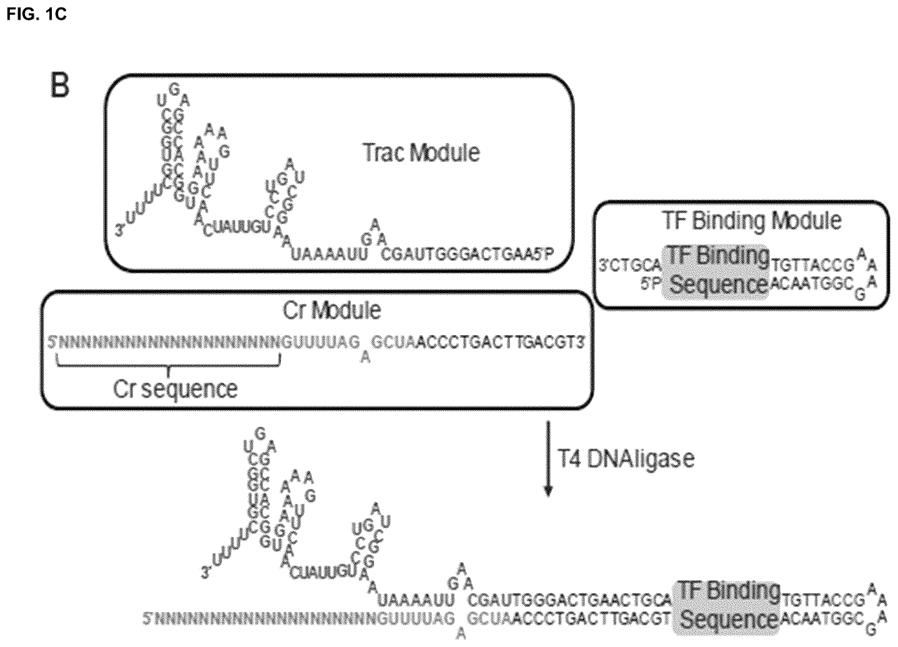

In one embodiment, with the use of the PGM, there is no need to edit the genome of the cells because the described system and methods can harness existing genetic material and metabolism and overcome safety concerns related to gene therapy and gene editing. In addition, the effects here are limited to only the relevant cell population in the relevant physiological environment and thus, any off-target effects can be minimized. Furthermore, the modular programmability of the system and methods may be applied to different gene targets and physiological actuators for addressing a range of injuries, diseases, and cell types. An example of the use of this platform, which is herein called Protege, may be seen in FIGS. 1A and 1B. The target gene is programmed by the sequence of the crRNA component of the guide nucleic acid and the transcription factor(s) to which transcriptional modulation responds is (are) programmed by the transcription factor response element(s) incorporated into the guide nucleic acid.

In one embodiment, the novel PGMs may function by recruiting endogenous transcription factors to the promoter region of genes targeted for modulation. Whereas extant designs for artificial transcription factors rely on the co-delivery of modules that affect gene transcription with modules that recognize the targeted gene promoter, the disclosed approach provides generalizability and control by harnessing transcription factors already present in the cell. FIGS. 1A and 1B show an overview of one possible embodiment of a programmable gene modulation platform. As the diagram indicates here, the transcription factor may be activated by a physiologic stimulus such as oxidative stress or growth factor signaling. In some embodiments, the PGM comprises a ribonucleoprotein complex composed of a disabled CRISPR-associated protein (e.g., dCas9) and a single guide chimeric nucleic acid (sgCNA), which includes a DNA hairpin that incorporates a binding site for the activated TF, a crispr (“cr”) RNA sequence and a trans-activating CRISPR (“tracr”) RNA sequence. The crRNA sequence is a sequence complementary to the target DNA, which may be typically 17-20 nucleotides long. The tracrRNA sequence serves as a binding scaffold for the Cas protein. This complex binds to a sequence of genomic DNA proximal to a target gene that is to be made responsive to the physiologic signal. The binding site is programmed by the crRNA sequence in the guide nucleic acid. Association of the activated TF with the bound dCas9 complex brings the TF in proximity to the target gene resulting in modulation of target gene transcription. In one embodiment, transcription is activated or enhanced. In other embodiments, transcription may be repressed or decreased. In one embodiment, an advantage of a design where the DNA hairpin caps an existing hairpin structure in the parent guide RNA (rather than being appended to the end) is that it provides cohesive double-stranded sites for ligation, facilitating the modular synthesis shown (FIG. 1C), which allows easy “mixing and matching” of genomic targets (defined by the crRNA module) and transcription factors (defined by the transcription factor binding module). One would not have to re-synthesize everything to swap out a module.

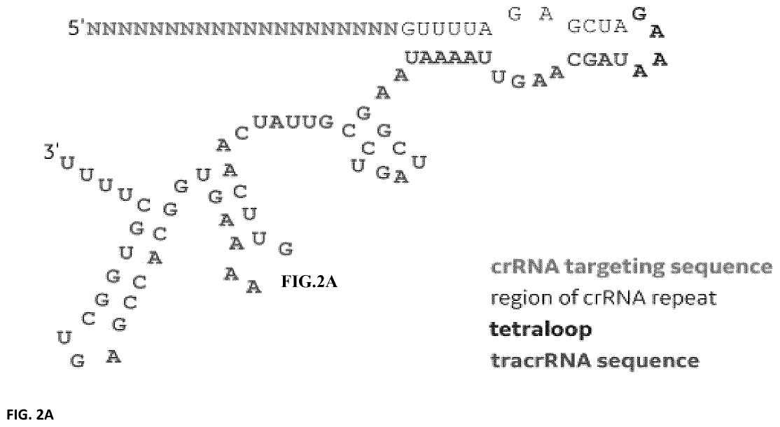

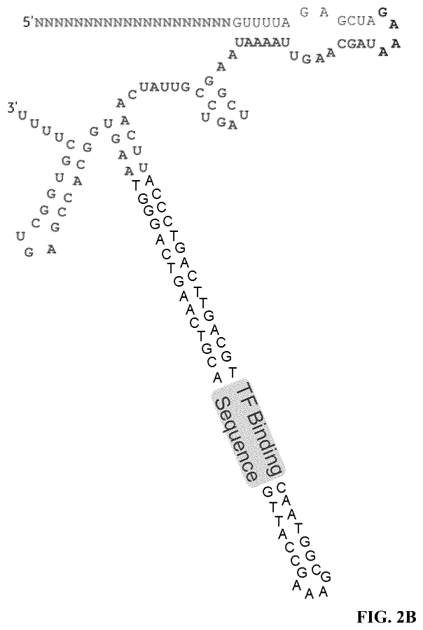

Accordingly, FIG. 2A is a schematic of a conventional structure of a sgRNA, while FIG. 2B and FIG. 2C show exemplary embodiments of a chimeric guide nucleic acid synthesis, noting modules included according to the disclosure. Compared to the sgRNA shown in FIG. 2B, in the sgRNA shown in FIG. 2C the chimeric guide nucleic acid DNA hairpin caps a different hairpin of the sgRNA from which the illustrated sgCNA is derived.

Also accordingly, in one embodiment, the disclosure provides a PGM that comprises two modules: (i) a genomic DNA binding module that defines the targeted gene and (ii) a transcription factor binding module that defines the transcription factor to be recruited. In some embodiments, the transcription factor binding module is a DNA duplex comprising the consensus binding sequence of the transcription factor to be linked to a therapeutic gene. In terms of whole molecules, the PGM is composed of the Cas protein and a single guide chimeric nucleic acid (sgCNA) comprising a crRNA sequence, a tracrRNA sequence, and a transcription factor binding site. For purposes of synthesis, the crRNA and tracrRNA sequences may be flanked by DNA sequences that serve the purposes of facilitating ligation by T4 DNA ligase. The crRNA/DNA fragment of the sgCNA is referred to herein as the Cr RNA module. The tracrRNA/DNA fragment of the sgCNA is referred to herein as the Tracr RNA module. The transcription factor binding site is also surrounded by additional DNA sequences that allow for the formation of a hairpin duplex. This hairpin duplex fragment is referred to herein as the Transcription Factor Binding Module. See FIG. 1B. In one embodiment, these three modules are each synthesized separately and then ligated to form the chimeric sgCNA molecule.

### The Genomic DNA Binding Module

In one embodiment, the genomic DNA binding functional module comprises a nuclease-defective Cas protein and the components of a single guide chimeric nucleic acid (sgCNA) that allow binding to the target DNA, specifically the RNA elements of the sgCNA. The genomic DNA binding module is designed to bind to genomic DNA proximal to the target therapeutic gene under natural conditions. When the transcription factor is activated by a stimulus (e.g., low oxygen), the PGM binds the activated transcription factor and delivers it to the target gene, modulating the expression of that gene.

In one embodiment, the DNA binding functional module comprises the two RNA portions of the chimeric guide nucleic acid comprising a crRNA sequence and a tracrRNA sequence. In one embodiment, the transcription factor binding module, a segment of DNA or RNA or modified nucleic acid that folds into a hairpin duplex, may be inserted between the crRNA and the tracrRNA sequences such that it does not interfere with the function of the guide RNA toward the target DNA recognition of the DNA binding module. In one embodiment, the chimeric guide nucleic acid is synthesized by ligation of three modules: a Trac Module, a Cr Module, and a TF binding module. See, e.g., FIG. 1C. RNA nucleotides are shown in blue and green bold font and DNA nucleotides are shown in black.

In one embodiment, the Trac and Cr Modules comprise RNA and DNA segments. In one embodiment, the DNA segments are complementary to each other and to a 3′ overhang of the TF binding module and the 5′ ends of the Trac and TF binding modules are phosphorylated to allow ligation of the Trac and Cr modules to the TF binding module. In one embodiment, the DNA segments on the Trac and Cr modules are sufficiently long for the three modules to comprise a substrate for T4 DNA ligase.

In one embodiment, the DNA segments comprise the sequence 5′-ACCCTGACTTGACGT-3′ (SEQ ID NO: 75) for the crRNA module and 5′-AAGTCAGGGT-3′ (SEQ ID NO: 76) for the tracrRNA module. In one embodiment, the modules are prepared by conventional solid phase oligonucleotide synthesis and purified by polyacrylamide gel electrophoresis. Again, because T4 DNA ligase does not efficiently ligate RNA to DNA, assembly of cr, tracr, and transcription factor binding components of the sgCNA by ligation with T4 DNA ligase may require an adaptor/linker segment attached on the cr and tracr components. It will be apparent to one skilled in the field that many different linker segment sequences will be effective. The DNA linker segments should be at least partially complementary and should, when hybridized, form a duplex with an overhang of at least one nucleotide and preferably at least four nucleotides. The overhang may base pair with a complementary overhang in a DNA duplex at the site of ligation to the DNA transcription factor binding component of the sgCNA. Either the 5′ or the 3′ end of the transcription factor binding component may be the recessed end of the overhang. Any Transcription Factor Binding module sequence with an overhang complementary to the overhang formed by the DNA segments of the Cr and Tracr modules can be ligated, enabling use of the same Cr and Tracr modules with different TF binding modules. The site of ligation on each strand may be at least five nucleotides and preferably at least ten nucleotides from the RNA nucleotides of the cr and tracr components of the sgCNA ligation reaction. Though many sequences can be used for the DNA linker segments, the sequences should be chosen such that they do not have significant internal base pairing or form other internal structures (such as G-quartets) within one linker segment or with the crRNA or tracrRNA components to which they are appended. This requirement can be determined by inspection or by use of nucleic acid folding tools that are widely known to those knowledgeable in the field. An example of one such tool is the program mfold.

In some embodiments, the crRNA comprises an RNA sequence complementary to a nucleic acid sequence in the promoter region of the gene of interest and each transcription factor binding site(s) of the PGM bind(s) to at least one endogenous transcription factor that is activated in the cell in response to the environmental signal(s) and then recognizes and binds to the transcription factor binding site of the PGM which is bound through the crRNA to the promoter of the gene of interest, thereby bringing the transcription factor into proximity with the gene of interest and activating or suppressing expression of the gene of interest in response to the environmental signal(s). In one embodiment, the target gene and crRNA sequence are selected from those of Table 1.

TABLE 1
Exemplary Target Genes and Respective Exemplary crRNA Sequences

| | | | crRNA | SEQ | | SEQ | |
|---|---|---|---|---|---|---|---|
| | Gene | Sequence | sequence | ID | Genomic DNA | ID | |
| Gene | product | Name | (5′-3′) | NO: | sequence | NO: | Strand |
| KL | klotho | klotho- | CGUCCACG | 6 | TGCAGGAC | 35 | Sense |
| | | S273 | AAACGUCC | | GTTTCGTGG | | |
| | | | UGCA | | ACG | | |
| KL | klotho | klotho- | GCAAUAAU | 7 | GGCTCGCA | 36 | Antisense |
| | | AS117 | UACCUGCG | | GGTAATTAT | | |
| | | | AGCC | | TGC | | |
| KL | klotho | klotho- | GAGCCGUG | 8 | CGAAACGT | 37 | Sense |
| | | S281 | CAGGACGU | | CCTGCACG | | |
| | | | UUCG | | GCTC | | |
| KL | klotho | klotho- | CAGGCGUC | 9 | ACGTCCGC | 38 | Antisense |
| | | AS228 | GCCCGCGG | | GGGCGACG | | |
| | | | ACGU | | CCG | | |
| KL | klotho | klotho- | UUAUUGCC | 10 | GCGGGCTC | 39 | Antisense |
| | | AS90 | AGCGGAGC | | CGCTGGCA | | |
| | | | CCGC | | ATAA | | |
| KL | klotho | klotho- | GUGCCUUU | 11 | CGGACGTC | 40 | Sense |
| | | S227 | CUCCGACG | | GGAGAAAG | | |
| | | | UCCG | | GCAC | | |
| KL | klotho | klotho- | CGGAGGCA | 12 | GCGAGCCG | 41 | Sense |
| | | S116 | GUCCCGGC | | GGACTGCC | | |
| | | | UCGC | | TCCG | | |
| KL | klotho | klotho- | UCCCGGGC | 13 | AGGGCGAG | 42 | Sense |
| | | S168 | ACCCCUCGC | | GGTGCCCG | | |
| | | | CCU | | GGA | | |
| KL | klotho | klotho- | CUGCCUCC | 14 | CAGTGCCA | 43 | Antisense |
| | | AS141 | GCCCUGGC | | GGGCGAGG | | |
| | | | ACUG | | CAG | | |
| KL | klotho | klotho- | GAGGGCGA | 15 | CCCGGGCA | 44 | Antisense |
| | | AS181 | GGGGUGCC | | CCCCTCGCC | | |
| | | | CGGG | | CTC | | |
| MIR196A1 | miRNA- | 196a-159 | ACAUCGGA | 16 | GGGTTCAG | 45 | antisense |
| | 196a | | AGAACUGA | | TTCTTCCGA | | |
| | | | ACCC | | TGT | | |
| MIR196A1 | miRNA- | 196a-38 | CUAGGUCA | 17 | CCAGTGAG | 46 | antisense |
| | 196a | | AGAGCUCA | | CTCTTGACC | | |
| | | | CUGG | | TAG | | |
| MIR196A1 | miRNA- | 196a-195 | GGAAUCUG | 18 | GTCCTGATA | 47 | sense |
| | 196a | | AGCUAUCA | | GCTCAGATT | | |
| | | | GGAC | | CC | | |
| MIR196A1 | miRNA- | 196a-167 | AGAACUGA | 19 | AGAGTCCT | 48 | antisense |
| | 196a | | ACCCAGGA | | GGGTTCAG | | |
| | | | CUCU | | TTCA | | |
| MIR196A1 | miRNA- | 196a-35 | GCUCUAGG | 20 | GTGAGCTCT | 49 | antisense |
| | 196a | | UCAAGAGC | | TGACCTAG | | |
| | | | UCAC | | AGC | | |
| MIR196A1 | miRNA- | 196a-37 | UCUAGGUC | 21 | CAGTGAGC | 50 | antisense |
| | 196a | | AAGAGCUC | | TCTTGACCT | | |
| | | | ACUG | | AGA | | |
| MIR196A1 | miRNA- | 196a-143 | GUUGCUAC | 22 | ATGTGTTGT | 51 | antisense |
| | 196a | | UAAACAAC | | TTAGTAGC | | |
| | | | ACAU | | AAC | | |
| MIR196A1 | miRNA- | 196a-128 | UGUGUUGU | 23 | AGTTGCTAC | 52 | sense |
| | 196a | | UUAGUAGC | | TAAACAAC | | |
| | | | AACU | | ACA | | |
| MIR196A1 | miRNA- | 196a-114 | GCAACUGG | 24 | CAACAGAA | 53 | sense |
| | 196a | | GUUCUUCU | | GAACCCAG | | |
| | | | GUUG | | TTGC | | |
| MIR196A1 | miRNA- | 196a-105 | UUCUUCUG | 25 | CCTCTTCCC | 54 | sense |
| | 196a | | UUGGGGAA | | CAACAGAA | | |
| | | | GAGG | | GAA | | |
| TGFB3 | TGFbeta- | TGFbeta3- | GCGAGUCC | 26 | CGTCGAAC | 55 | Antisense |
| | 3 | 152 | UCUCGUUC | | GAGAGGAC | | |
| | | | GACG | | TCGC | | |
| TGFB3 | TGFbeta- | TGFbeta3- | CAGCUACC | 27 | CTCGTTCGA | 56 | Sense |
| | 3 | 136 | ACGUCGAA | | CGTGGTAG | | |
| | | | CGAG | | CTG | | |
| TGFB3 | TGFbeta- | TGFbeta3- | GCGGACUG | 28 | CTCGCCAG | 57 | Sense |
| | 3 | 261 | ACAGCUGG | | CTGTCAGTC | | |
| | | | CGAG | | CGC | | |
| TGFB3 | TGFbeta- | TGFbeta3- | GAAUCACU | 29 | ACGTGTGG | 58 | Antisense |
| | 3 | 200 | CCUGCCAC | | CAGGAGTG | | |
| | | | ACGU | | ATTC | | |
| TGFB3 | TGFbeta- | TGFbeta3- | CGGACUGA | 30 | TCTCGCCAG | 59 | Sense |
| | 3 | 260 | CAGCUGGC | | CTGTCAGTC | | |
| | | | GAGA | | CG | | |
| TGFB3 | TGFbeta- | TGFbeta3- | GGCAGGAG | 31 | CTCTTGGAA | 60 | Sense |
| | 3 | 169 | UGAUUCCA | | TCACTCCTG | | |
| | | | AGAG | | CC | | |
| TGFB1 | TGFbeta- | TGFbeta 1- | CGGGUGAU | 32 | CAGCGCAT | 61 | Antisense |
| | 1 | 268 | CCAGAUGC | | CTGGATCA | | |
| | | | GCUG | | CCCG | | |
| TGFB1 | TGFbeta- | TGFbeta 1- | CGGAUUAA | 33 | GGCGGAGA | 62 | Sense |
| | 1 | 304 | GCCUUCUC | | AGGCTTAA | | |
| | | | CGCC | | TCCG | | |
| TGFB1 | TGFbeta- | TGFbeta 1- | GAGCCCGC | 34 | CATCTCGCG | 63 | Sense |
| | 1 | 125 | CCACGCGA | | TGGGCGGG | | |
| | | | GAUG | | CTC | | |

In one embodiment, the DNA binding module comprises a ribonucleoprotein complex that further comprises a CRISPR-associated protein such as Cas9 that has been mutated to eliminate its DNA cleavage activity. In one embodiment, the tracrRNA binds to dCas9. In another embodiment, the tracrRNA binds to any other nuclease-defective DNA binding protein (DNAbp). In some embodiments, the DNAbp is selected from nuclease-defective Cas9, Cas12e, Cas12d, Cas12a, Cas12b1, Cas13a, Cas12c, ArgonauteCas12b2, Cas13a, Cas12c, Cas12d, Cas12e, Cas12h, Cas12i, Cas12g, Cas12f (Cas14), Cas12f1, Cas12j (Casǐ), and Argonaute.

### The Transcription Factor Binding Module

In one embodiment, the PGM recruits an endogenous transcription factor(s) to the gene of interest when the endogenous transcription factor(s) has/have been activated in response to an environmental signal(s), thereby modulating gene expression in response to the environmental signal(s), in a cell-specific manner. In one embodiment, the PGM comprises at least one TFBM/TFBS. The use or two of more TFBSs in the same PGM may be used to increase specificity or activity. In one embodiment, the TF binding module is a DNA hairpin incorporating one or more TF binding sequences (TFBS) in its double-stranded sequence. In one embodiment, the loop sequence of this hairpin is the exceptionally stable GAAA tetraloop, which promotes proper folding of the hairpin and of the full guide nucleic acid. In one embodiment, a 3′ overhang and a 5′ phosphate (5′P) allow ligation of this module to the Trac and Cr modules.

In one embodiment, at least one of the TFBSs in the PGM is also present in the target gene. In one embodiment, at least one of the TFBS in the PGM is not an endogenous TFBS in the target gene. In one embodiment, the transcription factor is selected from forkhead transcription factors, nuclear receptors, POU-domain proteins, SMAD, preferably Nrf2, FOX01, NF-kB, USF2, NFAT, EGR1, STAT3, and SREBP. In one embodiment, the transcription factor is Nrf2. In one embodiment, the transcription factor is selected from those listed in Table 2.

TABLE 2
Exemplary Transcription Factor Binding Sites

| Transcription | | |
|---|---|---|
| Factor | Response element (N = any base) | Reference |
| p53 | 5′- | el-Deiry WS, Kern SE, Pietenpol |
| | (A/G)(A/G)(A/G)C(A/T)(A/T)G(C/T)(C/T) | JA, Kinzler KW, Vogelstein B |
| | C/T)(N)₀₋₁₃₎(A/G)(A/G)(A/G)C(A/T)(A/T) | (1992) Definition of a consensus |
| | G(C/T)(C/T)(C/T)-3′ (SEQ ID NO: 1) | binding site for p53. Nat Genet |
| | | 1:45-49 |
| ER | 5′-GGTCANNNTGACC-3′ (SEQ ID NO: 2) | Klein-Hitpass L, Ryffel G. U., |
| | | Heitlinger E., Cato A. C. Nucleic |
| | | Acids Res. 1988; 16: 647-663 |
| MYC/MAX | 5′-CACGTG-3′ | Blackwell, T. K., Kretzner, L., |
| | | Blackwood, E. M., Eisenman, R. |
| | | N., & Weintraub, H. (1990). |
| | | Sequence-specific DNA binding |
| | | by the c-Myc protein. Science, |
| | | 250(4984), 1149-1151. |
| AP1 | 5′-TGA(G/C)TCA-3′ | Angel, P., Imagawa, M., Chiu, |
| | | R., Stein, B., Imbra, R. J., |
| | | Rahmsdorf, H. J., . . . & Karin, M. |
| | | (1987). Phorbol ester-inducible |
| | | genes contain a common cis |
| | | element recognized by a TPA- |
| | | modulated trans-acting factor. |
| | | Cell, 49(6), 729-739. |
| SP1 | 5′-(G/T)GGGGGG(G/A)(G/A)(C/T)-3′ | 3Kadonaga, J. T., Jones, K. A., & |
| | (SEQ ID NO: 3) | Tjian, R. (1986). Promoter- |
| | | specific activation of RNA |
| | | polymerase II transcription by |
| | | Sp1. Trends in Biochemical |
| | | Sciences, 11(1), 20-23. |
| NFKB | 5′-GGGNNNNNCC-3′ | Chen, F. E., Ghosh G. Regulation |
| | | of DNA binding by Rel/NF-KB |
| | | transcription factors: structural |
| | | views. Oncogene. 1999; 18:6845-6852 |
| CREB | 5′-TGACGTCA-3 | Carlezon Jr, W. A., Duman, R. S., |
| | | & Nestler, E. J. (2005). The many |
| | | faces of CREB. Trends in |
| | | neurosciences, 28(8), 436-445. |
| MYB | 5′-(C/T)AAC(G/T)G-3′ | Ciciro, Y., & Sala, A. (2021). |
| | | MYB oncoproteins: emerging |
| | | players and potential |
| | | therapeutic targets in human |
| | | cancer. Oncogenesis, 10(2), 1-15. |
| STAT1 | 5′-TTNCNNNAA-3′ | Chon, Sook Y., Hamdy H. |
| | | Hassanain, and Sohan L. Gupta. |
| | | “Cooperative role of interferon |
| | | regulatory factor 1 and p91 |
| | | (STAT1) response elements in |
| | | interferon-v-inducible |
| | | expression of human |
| | | indoleamine 2, 3-dioxygenase |
| | | gene.” Journal of Biological |
| | | Chemistry 271.29 (1996): |
| | | 17247-17252. |
| ETS1 | 5′-GGA(A/T)-3′ | Shiu, Yan-Ting, and Edgar A. |
| | | Jaimes. “Transcription factor |
| | | ETS-1 and reactive oxygen |
| | | species: role in vascular and |
| | | renal injury.” Antioxidants 7.7 |
| | | (2018): 84. |
| SMAD2/3 | 5′-GTCTAGAC-3 | A comparative analysis of |
| | | Smad-responsive motifs |
| | | identifies multiple regulatory |
| | | inputs for TGF-β transcriptional |
| | | activation Itoh Y., Koinuma D., |
| | | Omata C., Ogami T., Motizuki |
| | | M., Yaguchi S.-I., Itoh T., ( . . . ), |
| | | Miyazawa K. (2019) Journal of |
| | | Biological Chemistry, 294 (42), |
| | | pp. 15466-15479. |
| NRF2 | 5′-ATGACTCAGCA-3′ (SEQ ID NO: 5) | Malhotra D, Portales-Casamar E, |
| | | Singh A, et al. Global mapping of |
| | | binding sites for Nrf2 identifies |
| | | novel targets in cell survival |
| | | response through ChIP-Seq |
| | | profiling and network analysis. |
| | | Nucleic Acids Res. |
| | | 2010; 38(17):5718-5734 |
| FOXO1 | 5′-TT(G/A)TTTAC-3′ | Furuyama, T., Nakazawa, T., |
| | | Nakano, I., & Mori, N. (2000). |
| | | Identification of the differential |
| | | distribution patterns of mRNAs |
| | | and consensus binding |
| | | sequences for mouse DAF-16 |
| | | homologues. Biochemical |
| | | Journal, 349(2), 629-634. |
| USF2 | 5′-CACGTG-3′ | Luo, X., & Sawadogo, M. (1996). |
| | | Antiproliferative properties of |
| | | the USF family of helix-loop- |
| | | helix transcription factors. |
| | | Proceedings of the National |
| | | Academy of Sciences, 93(3), |
| | | 1308-1313. |
| EGR1 | 5′-GCGTGGGCG-3′ | Christy B, Nathans D |
| | | (November 1989). “DNA binding |
| | | site of the growth factor- |
| | | inducible protein Zif268”. |
| | | Proceedings of the National |
| | | Academy of Sciences of the |
| | | United States of America. 86 |
| | | (22): 8737-41. |
| STAT3 | 5′-TTCCCGGAA-3′ | Cocchiola, R., Grillo, C., Altieri, |
| | | F., Chichiarelli, S., Turano, C., & |
| | | Eufemi, M. (2014). Upregulation |
| | | of TPX2 by STAT3: identification |
| | | of a novel STAT3 binding site. |
| | | PloS one, 9(11), e113096. |
| SREBP | 5′-TCACNCCAC-3′ | Smith, J. R., Osborne, T. F., |
| | | Goldstein, J. L., & Brown, M. S. |
| | | (1990). Identification of |
| | | nucleotides responsible for |
| | | enhancer activity of sterol |
| | | regulatory element in low |
| | | density lipoprotein receptor |
| | | gene. Journal of Biological |
| | | Chemistry, 265(4), 2306-2310. |
| NFAT | 5′-(A/T)GGAAAN(A/T/C)N-3′ | Rao, A., Luo, C., & Hogan, P. G. |
| | | (1997). Transcription factors of |
| | | the NFAT family: regulation and |
| | | function. Annual review of |
| | | immunology, 15(1), 707-747. |

In one embodiment, the transcription factor is selected from those listed in public transcription factor databases, such as the TRRUST database and the Dorothea database. In one embodiment, the specific sequence to which the TF binds, also known as a TF motif, may be selected from TF motif databases such as JASPAR, HOCOMOCO, CIS-BP, and others (see, e.g., Stormo, G. D. (2015). DNA motif databases and their uses. Current Protocols in Bioinformatics, 51, 2.15.1-2.15.6). These motifs may also be used to predict TFBSs in the genome using tools like PWMscan (Ambrosini, G., Groux, R., & Bucher, P. (2018). PWMScan: A fast tool for scanning entire genomes with a position-specific weight matrix. Bioinformatics, 34, 2483-2484), or MOODS (Korhonen, J., Martinmaki, P., Pizzi, C., Rastas, P., & Ukkonen, E. (2009). MOODS: fast search for position weight matrix matches in DNA sequences. Bioinformatics, 25 (23), 3181-3182). In addition, recent advances in TF-mapping technology combined with deep-learning algorithms to predict TF binding sites have been used successfully to predict direct binding for some TFs (Avsec, Ž., Weilert, M., Shrikumar, A., Krueger, S., Alexandari, A., Dalal, K., Fropf, R., McAnany, C., Gagneur, J., Kundaje, A., & Zeitlinger, J. (2021). Base-resolution models of transcription-factor binding reveal soft motif syntax. Nature Genetics, 53, 354-366). Moreover, a profile of TFs across thousands of cell types is available (e.g., Moore, J. E., Purcaro, M. J., Pratt, H. E., Epstein, C. B., Shoresh, N., Adrian, J., Kawli, T., Davis, C. A., Dobin, A., Kaul, R., Halow, J., van Nostrand, E. L., Freese, P., Gorkin, D. U., Shen, Y., He, Y., Mackiewicz, M., Pauli-Behn, F., Williams, B. A . . . . Weng, Z. (2020). Expanded encyclopaedias of DNA elements in the human and mouse genomes. Nature, 583, 699-710) and databases collecting experimentally measured TF binding sites (e.g., REMAP, ChIP-Atlas, or GTRD) may be used to select TF binding in specific cell types.

In one embodiment, the TF is selected from those listed in Table 3.

TABLE 3
Exemplary Transcription Factors for the PGM of the disclosure

| Forkhead | | | |
|---|---|---|---|
| transcription | POU-Domain | | |
| factors | Proteins | SMAD | Nuclear Receptors |
| Forkhead box b1 | Oct-2 | SMAD | Small heterodimer |
| Forkhead box C1 | Oct-4 | Medea gene | partner |
| Forkhead box d1 | Octamer transcription | I-SMAD | Constitutive |
| Forkhead Box | factor | R-SMAD | androstane receptor |
| Protein L2 | Pituitary-specific | Mothers against | Daf-12 |
| Forkhead box | positive transcription | decapentaplegic | Ecdysone receptor |
| protein O1 | factor 1 | | Liver X receptor |
| FOXA1 | POU2F1 | | Peroxisome |
| FOXA2 | POU2F3 | | proliferator-activated |
| FOXA3 | POU3F1 | | receptor |
| FOXC2 | POU3F2 | | Pregnane X receptor |
| FOXD3 | POU3F4 | | Rev-Erb |
| FOXD4 | POU4F1 | | Thyroid hormone |
| FOXE1 | POU4F2 | | receptor |
| FOXE3 | POU4F3 | | Androgen receptor |
| FOXF1 | | | Estrogen receptor |
| FOXF2 | | | Estrogen-related |
| FOXG1 | | | receptor |
| FOXH1 | | | Glucocorticoid |
| FOXI1 | | | receptor |
| FOXI3 | | | Mineralocorticoid |
| FOXJ1 | | | receptor |
| FOXJ2 | | | Liver receptor |
| FOXK1 | | | homolog-1 |
| FOXK2 | | | Steroidogenic factor |
| FOXL2 | | | 1 |
| FOXM1 | | | |
| FOXN1 | | | |
| FOXN3 | | | |
| FOXO3 | | | |
| FOXO4 | | | |
| FOXO6 | | | |
| FOXP1 | | | |
| FOXP2 | | | |
| FOXP3 | | | |
| FOXP4 | | | |
| FOXQ1 | | | |
| FOXS1 | | | |

In one embodiment, the TF binding site (TFB/TFBS) is separated from the loop by eight base pairs to ensure the structure of the TF binding site is not distorted from its native TF-binding conformation. In some embodiments, the PGM comprises more than one TF binding site.

In one embodiment, the PGM modules are assembled into any one of the following configurations:
5′-crRNA-TFBS-tracrRNA-3′;
5′-crRNA-tracrRNA′-TFBS-tracrRNA″-3′ (wherein the TFBSD is integrated anywhere within the sequence of the tracrRNA, including as an extension to one or more of its hairpin structures);
5′-crRNA-tracrRNA-TFBS-3′;
5′-crRNA-TFBS-tracrRNA′-TFBS-tracrRNA″-3′;
5′-crRNA-TFBS-tracrRNA-TFBS-3′;
5′-crRNA-tracrRNA′-TFBS-tracrRNA″-TFBS-3′;
5′-crRNA-TFBS-tracrRNA′-TFBS-tracrRNA″-TFBS-3′;
5′-crRNA-TFBS-tracrRNA′-TFBS-tracrRNA″-TFBS-tracrRNA′″-3′;
5′-crRNA-TFBS-tracrRNA′-TFBS-tracrRNA″-TFBS-tracrRNA′″-TFBS-tracrRNA″″-3′;
5′-crRNA-tracrRNA′-TFBS-tracrRNA″-TFBS-tracrRNA′″-TFBS-3′;
5′-crRNA-tracrRNA′-TFBS-tracrRNA″-TFBS-tracrRNA′″-TFBS-tracrRNA″″-TFBS-3′;
5′-crRNA-TFBS-tracrRNA-TFBS-tracrRNA-TFBS-tracrRNA-TFBS-3′;
5′-crRNA-TFBS-tracrRNA-TFBS-tracrRNA-TFBS-tracrRNA-TFBS-tracrRNA-TFBS-3′;
5′-crRNA-tracrRNA′-TFBS-tracrRNA″-TFBS-tracrRNA′″-3′;
5′-crRNA-tracrRNA′-TFBS-tracrRNA″-TFBS-tracrRNA′″-TFBS-tracrRNA″″-3′;

In these configurations, tracRNA′, tracrRNA″, tracrRNA′″, and tracrRNA′″ are successive segments of the complete tracrRNA sequence. The terms TB binding site vs TFBS vs TFB are all used interchangeably.

### PGM Delivery

In one embodiment, the PGM, or an individual component thereof (i.e., protein component, sgRNA component), is delivered to a subject enterally. In one embodiment, the PGM, or an individual component thereof, is delivered to a subject parenterally. In one embodiment, the PGM, or an individual component thereof, is delivered topically. In one embodiment, the PGM, or an individual component thereof, is delivered topically, subcutaneously, intraocularly, intravitreally, subretinally, intravenously (IV), intracerebro-ventricularly, intramuscularly, intrathecally (IT), intracisternally, intraperitoneally, via inhalation, or by direct injection to one or more cells, tissues, or organs. In some embodiments, the PGM delivery is targeted to a specific tissue or cell type. In one embodiment, the PGM is delivered to the cell via nucleic acid transfection (including electroporation, liposomal delivery, etc.) or viral transduction. In some embodiments, the PGM is delivered with lipid nanoparticles. In other embodiments, the PGM is delivered with liposomes. In other embodiments, the PGM is delivered with polymeric nanoparticles, such as polymersomes, dendrimers, polymer micelles, or polymer nanospheres. In other embodiments, the PGM is delivered with inorganic nanoparticles, such as silica nanoparticles, iron oxide nanoparticles, or gold nanoparticles.

In one embodiment, the PGM is delivered to the cell via cell-penetrating peptides, chemical moieties that mediate uptake into cells by binding to one or more receptors on the cell surface, or cell-type specific peptidic delivery agents (including antibodies and peptides derived from combinatorial libraries, and peptides discovered for selective internalization and/or subcellular localization by phage display biopanning). In one embodiment, the PGM is delivered with peptides discovered for selective internalization and/or subcellular localization by phage display biopanning with the molecular guidance system platform described in PCT International Publications WO2019014199, WO2019014190, and WO2021066931. In one embodiment, the PGM is delivered to the relevant cell type using a peptide or peptide derivative that mediates cell-specific uptake of bound cargo, such as peptides discovered for selective internalization and/or subcellular localization by phage display biopanning. In one such embodiment, the PGM is encapsulated in a lipid nanoparticle or liposome that displays the cell-selective peptide or peptide derivative on its surface. Lipid nanoparticles or liposomes of various formulations can be used in this embodiment, including lipid nanoparticles or liposomes that bear polyethylene glycol on their surface to minimize immunogenicity. In one embodiment, the lipid nanoparticle may contain cationic or ionizable lipid compounds to complex with the negatively charged PGM and aid endosomal escape. In another embodiment, the cell-type selective peptide or peptide derivative is conjugated directly to the PGM, either by conjugation to the protein component or to the guide RNA component.

### Exemplary Applications of the Protege Platform

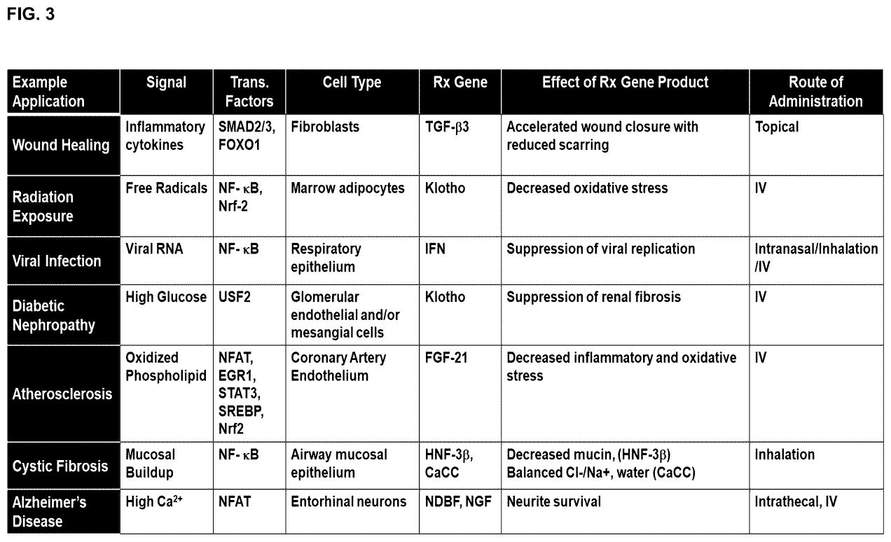

In addition to being useful for modulating gene expression in vitro, the PROTEGE Platform may be used in the treatment of any disease or disorder that benefits from the upregulation or downregulation of the expression of a specific target gene. FIG. 3 provides various non-limiting examples of different applications of the PROTEGE Platform. The PGM is designed to modulate gene expression in response to one or more intracellular or extracellular environmental signals. In one embodiment, the environmental signal is a physiological signal. In one embodiment, the environmental signal is associated with a pathological condition of disease, cellular stress, and/or injury. In one embodiment, the signal is an intrinsic signal such as one associated with development and differentiation.

In one embodiment, the signal is a physical signal. In one embodiment, the signal is a light signal (e.g., UV light), ionizing radiation, heat/temperature, hyperosmotic or hypoosmotic conditions. In some embodiments, the signal is a mechanical signal. In some embodiments, the signal is selected from pressure (e.g., touch), movement of sound waves, and blood pressure. In one embodiment, the signal is a chemical signal. In some embodiments, the chemical signal is a growth factor, a cytokine, a chemokine, cyclic AMP, a hormone, a neurotransmitter, an extracellular matrix component, a bacterial antigen, a viral antigen, a lipopolysaccharide, gas levels (e.g., oxygen levels, nitric oxide levels), ion levels (e.g., calcium levels, sodium levels), pH, a reactive oxygen species, a heavy metal, oxidized LDL, free radicals. In one embodiment, the signal is sensed by a receptor. In some embodiments, the receptor is an intracellular receptor (e.g., cytoplasmic, nuclear). In some embodiments, the receptor is a cell-surface/extracellular/transmembrane receptor. In some embodiments, the membrane receptor is selected from a G-protein-coupled receptor, an ion channel receptor, and enzyme-linked receptor. In some embodiments, the signal triggers a signal transduction cascade. In some embodiments, the signal transduction cascade triggers activation of a transcription factor to modulate gene expression. In some embodiments, the receptor is a transcription factor itself, such as nuclear receptors for lipid-soluble ligands (e.g., steroid hormones). In one example, the receptor/transcription factor is an estrogen receptor or glucocorticoid receptor, which reside in the cytoplasm until binding to their ligand allows translocation to the nucleus and expression of target genes.

Non-limiting examples of well-known signaling cascades that lead to the activation of TFs are TGFbeta signaling leading to activation the of SMAD family TFs, Jak-STAT signaling activating the STAT TFs, Erbb2 signaling typically activating Jun and Myc, Hippo signaling targeting the TEA-domain-containing (TEAD) family (TEAD1-TEAD4) of TFs, and Notch signaling that induces dissociation of DNA-bound RBPJ from a corepressor complex and recruitment of a coactivator complex instead. Examples of TFs that are inactivated by signaling include the FOXO family, a subclass of Forkhead TFs. In the absence of insulin, FOXO TFs are bound to DNA and activate gene expression. Upon insulin presence, FOXO TFs are phosphorylated by kinases downstream of the PI3K-AKT signaling pathway, which leads to exclusion of TFs from the nucleus and hence repression of their target gene.

In one embodiment, the signal is associated with a physiological condition. In one embodiment, the signal is associated with a pathological condition of disease, cellular stress, or injury such as: wound healing, radiation exposure, viral or bacterial infection, sepsis, diabetic nephropathy, atherosclerosis, cystic fibrosis, Alzheimer's disease, oxidative stress, ischemia-reperfusion injury, inflammation, cancer, anti-cancer agent resistance, a genetic disease, or any other proliferative disease or disorder, inflammatory disease or disorder, autoimmune disease or disorder, liver disease or disorder, spleen disease or disorder, lung disease or disorder, hematological disease or disorder, neurological disease or disorder, gastrointestinal (GI) tract disease or disorder, genitourinary disease or disorder, infectious disease or disorder, musculoskeletal disease or disorder, endocrine disease or disorder, metabolic disease or disorder, immune disease or disorder, central nervous system (CNS) disease or disorder, neurological disease or disorder, ophthalmic disease or disorder, or a cardiovascular disease or disorder.

In one embodiment, the anti-cancer agent to which resistance results in a signal that activates a transcription factor encompasses biotherapeutic anti-cancer agents as well as chemotherapeutic agents. Exemplary biotherapeutic anti-cancer agents include, but are not limited to, interferons, cytokines (e.g., tumor necrosis factor, interferon a, interferon g), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g. HERCEPTIN (trastuzumab), T-DM1, AVASTIN (bevacizumab), ERBITUX (cetuximab), VECTIBIX (panitumumab), RITUXAN (rituximab), BEXXAR (tositumomab)). Exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photo sensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB 2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunombicin, doxorubicin, pegylated liposomal doxorubicin, idarubicin, epirubicin, pirarubicin, zombicin, mitoxantrone), MDR inhibitors (e.g. verapamil), Ca2+ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genentech), SF1126 (Semafoe), and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

In one embodiment, the PGM is used to treat an “autoimmune disease or disorder,” which refers to a disease or disorder arising from an inappropriate immune response of the body of a subject against substances and tissues normally present in the body. In other words, the immune system mistakes some part of the body as a pathogen and attacks its own cells. This disfunction may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, systemic lupus erythematosis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

In one embodiment, the PGM is used to treat “cancer,” which refers to a class of diseases characterized by the development of abnormal cells that proliferate uncontrollably and have the ability to infiltrate and destroy normal body tissues. See e.g., Stedman's Medical Dictionary, 25th ed.; Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendoctrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva.

In one embodiment, the PGM is used to treat a “genetic disease or disorder,” which refers to a disease or disorder caused by one or more abnormalities in the genome of a subject, such as a disease that is present from birth of the subject. Genetic diseases or disorders may be heritable and may be passed down from the parents' genes. A genetic disease or disorder may also be caused by mutations or changes of the DNAs and/or RNAs of the subject. In such cases, the genetic disease or disorder will be heritable if it occurs in the germline. Exemplary genetic diseases or disorders include, but are not limited to, Aarskog-Scott syndrome, Aase syndrome, achondroplasia, acrodysostosis, addiction, adreno-leukodystrophy, albinism, ablepharon-macrostomia syndrome, alagille syndrome, alkaptonuria, alpha-1 antitrypsin deficiency, Alport's syndrome, Alzheimer's disease, asthma, autoimmune polyglandular syndrome, androgen insensitivity syndrome, Angelman syndrome, ataxia, ataxia telangiectasia, atherosclerosis, attention deficit hyperactivity disorder (ADHD), autism, baldness, Batten disease, Beckwith-Wiedemann syndrome, Best disease, bipolar disorder, brachydactyl), breast cancer, Burkitt lymphoma, chronic myeloid leukemia, Charcot-Marie-Tooth disease, Crohn's disease, cleft lip, Cockayne syndrome, Coffin Lowry syndrome, colon cancer, congenital adrenal hyperplasia, Cornelia de Lange syndrome, Costello syndrome, Cowden syndrome, craniofrontonasal dysplasia, Crigler-Najjar syndrome, Creutzfeldt-Jakob disease, cystic fibrosis, deafness, depression, diabetes, diastrophic dysplasia, DiGeorge syndrome, Down's syndrome, dyslexia, Duchenne muscular dystrophy, Dubowitz syndrome, ectodermal dysplasia Ellis-van Creveld syndrome, Ehlers-Danlos, epidermolysis bullosa, epilepsy, essential tremor, familial hypercholesterolemia, familial Mediterranean fever, fragile X syndrome, Lriedreich's ataxia, Gaucher disease, glaucoma, glucose galactose malabsorption, glutaricaciduria, gyrate atrophy, Goldberg Shprintzen syndrome (velocardiofacial syndrome), Gorlin syndrome, Hailey-Hailey disease, hemihypertrophy, hemochromatosis, hemophilia, hereditary motor and sensory neuropathy (HMSN), hereditary non polyposis colorectal cancer (HNPCC), Huntington's disease, immunodeficiency with hyper-IgM, juvenile onset diabetes, Klinefelter's syndrome, Kabuki syndrome, Leigh's disease, long QT syndrome, lung cancer, malignant melanoma, manic depression, Marfan syndrome, Menkes syndrome, miscarriage, mucopolysaccharide disease, multiple endocrine neoplasia, multiple sclerosis, muscular dystrophy, myotrophic lateral sclerosis, myotonic dystrophy, neurofibromatosis, Niemann-Pick disease, Noonan syndrome, obesity, ovarian cancer, pancreatic cancer, Parkinson's disease, paroxysmal nocturnal hemoglobinuria, Pendred syndrome, peroneal muscular atrophy, phenylketonuria (PKU), polycystic kidney disease, Prader-Willi syndrome, primary biliary cirrhosis, prostate cancer, REAR syndrome, Refsum disease, retinitis pigmentosa, retinoblastoma, Rett syndrome, Sanfilippo syndrome, schizophrenia, severe combined immunodeficiency, sickle cell anemia, spina bifida, spinal muscular atrophy, spinocerebellar atrophy, sudden adult death syndrome, Tangier disease, Tay-Sachs disease, thrombocytopenia absent radius syndrome, Townes-Brocks syndrome, tuberous sclerosis, Turner syndrome, Usher syndrome, von Hippel-Lindau syndrome, Waardenburg syndrome, Weaver syndrome, Wemer syndrome, Williams syndrome, Wilson's disease, xeroderma piginentosum, and Zellweger syndrome.

In one embodiment, the PGM is used to treat an “hematological disease or disorder,” which includes a disease or disorder which affects a hematopoietic cell or tissue. Hematological diseases or disorders include diseases or disorder associated with aberrant hematological content and/or function. Examples of hematological diseases or disorders include diseases resulting from bone marrow irradiation or chemotherapy treatments for cancer, diseases such as pernicious anemia, hemorrhagic anemia, hemolytic anemia, aplastic anemia, sickle cell anemia, sideroblastic anemia, anemia associated with chronic infections such as malaria, trypanosomiasis, HTV, hepatitis virus or other viruses, myelophthisic anemias caused by marrow deficiencies, renal failure resulting from anemia, anemia, polycythemia, infectious mononucleosis (EVI), acute non-lymphocytic leukemia (ANLL), acute myeloid leukemia ((AML), acute promyelocytic leukemia (APL), acute myelomonocytic leukemia (AMMoL), polycythemia vera, lymphoma, acute lymphocytic leukemia (ALL), chronic lymphocytic leukemia, Wilm's tumor, Ewing's sarcoma, retinoblastoma, hemophilia, disorders associated with an increased risk of thrombosis, herpes, thalassemia, antibody-mediated disorders such as transfusion reactions and erythroblastosis, mechanical trauma to red blood cells such as micro-angiopathic hemolytic anemias, thrombotic thrombocytopenia purpura and disseminated intravascular coagulation, infections by parasites such as Plasmodium, chemical injuries from, e.g., lead poisoning, and hypersplenism.

In one embodiment, the PGM is used to treat an “inflammatory disease or disorder” and “inflammatory condition” are used interchangeably herein, which refer to a disease or disorder or condition caused by, resulting from, or resulting in inflammation. Inflammatory diseases or disorders and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, haemolytic autoimmune anaemia), asthma, arteritis (e.g., polyarteritis, temporal arteritis, periarteritis nodosa, Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruritus (itch)), endometriosis, Guillain-Barre syndrome, infection, ischaemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopeniarpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), lupus, multiple sclerosis, morphea, myeasthenia gravis, myocardial ischemia, nephrotic syndrome, pemphigus vulgaris, pernicious aneaemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, schleroderma, scierodoma, sarcoidosis, spondyloarthopathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatistis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease).

In one embodiment, the PGM is used to treat a “liver disease or disorder” or “hepatic disease,” which refers to damage to or a disease of the liver. Non-limiting examples of liver disease or disorder include intrahepatic cholestasis (e.g., alagille syndrome, biliary liver cirrhosis), fatty liver (e.g., alcoholic fatty liver, Reye's syndrome), hepatic vein thrombosis, hepatolenticular degeneration (i.e., Wilson's disease), hepatomegaly, liver abscess (e.g., amebic liver abscess), liver cirrhosis (e.g., alcoholic, biliary, and experimental liver cirrhosis), alcoholic liver diseases (e.g., fatty liver, hepatitis, cirrhosis), parasitic liver disease (e.g., hepatic echinococcosis, fascioliasis, amebic liver abscess), jaundice (e.g., hemolytic, hepatocellular, cholestatic jaundice), cholestasis, portal hypertension, liver enlargement, ascites, hepatitis (e.g., alcoholic hepatitis, animal hepatitis, chronic hepatitis (e.g., autoimmune, hepatitis B, hepatitis C, hepatitis D, drug induced chronic hepatitis), toxic hepatitis, viral human hepatitis (e.g., hepatitis A, hepatitis B, hepatitis C, hepatitis D, hepatitis E), granulomatous hepatitis, secondary biliary cirrhosis, hepatic encephalopathy, varices, primary biliary cirrhosis, primary sclerosing cholangitis, hepatocellular adenoma, hemangiomas, bile stones, liver failure (e.g., hepatic encephalopathy, acute liver failure), angiomyolipoma, calcified liver metastases, cystic liver metastases, fibrolamellar hepatocarcinoma, hepatic adenoma, hepatoma, hepatic cysts (e.g., Simple cysts, Polycystic liver disease, hepatobiliary cystadenoma, choledochal cyst), mesenchymal tumors (mesenchymal hamartoma, infantile hemangioendothelioma, hemangioma, peliosis hepatis, lipomas, inflammatory pseudotumor), epithelial tumors (e.g., bile duct hamartoma, bile duct adenoma), focal nodular hyperplasia, nodular regenerative hyperplasia, hepatoblastoma, hepatocellular carcinoma, cholangiocarcinoma, cystadenocarcinoma, tumors of blood vessels, angiosarcoma, Karposi's sarcoma, hemangioendothelioma, embryonal sarcoma, fibrosarcoma, leiomyosarcoma, rhabdomyosarcoma, carcinosarcoma, teratoma, carcinoid, squamous carcinoma, primary lymphoma, peliosis hepatis, erythrohepatic porphyria, hepatic porphyria (e.g., acute intermittent porphyria, porphyria cutanea tarda), and Zellweger syndrome.

In one embodiment, the PGM is used to treat a “lung disease or disorder” or “pulmonary disease or disorder,” which refers to a disease or disorder of the lung. Examples of lung diseases or disorders include, but are not limited to, bronchiectasis, bronchitis, bronchopulmonary dysplasia, interstitial lung disease, occupational lung disease, emphysema, cystic fibrosis, acute respiratory distress syndrome (ARDS), severe acute respiratory syndrome (SARS), asthma (e.g., intermittent asthma, mild persistent asthma, moderate persistent asthma, severe persistent asthma), chronic bronchitis, chronic obstructive pulmonary disease (COPD), emphysema, interstitial lung disease, sarcoidosis, asbestosis, aspergilloma, aspergillosis, pneumonia (e.g., lobar pneumonia, multilobar pneumonia, bronchial pneumonia, interstitial pneumonia), pulmonary fibrosis, pulmonary tuberculosis, rheumatoid lung disease, pulmonary embolism, and lung cancer (e.g., non-small-cell lung carcinoma (e.g., adenocarcinoma, squamous-cell lung carcinoma, large-cell lung carcinoma), small-cell lung carcinoma).

In one embodiment, the PGM is used to treat a “neurological disease or disorder,” which refers to any disease or disorder of the nervous system, including diseases or disorders that involve the central nervous system (brain, brainstem, and cerebellum), the peripheral nervous system (including cranial nerves), and the autonomic nervous system (parts of which are located in both central and peripheral nervous system). Neurodegenerative diseases or disorders refer to a type of neurological disease or disorder marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases or disorders include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle and neuromuscular junctions. Addiction and mental illness include, but are not limited to, bipolar disorder and schizophrenia, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Amold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTFV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Fafora disease; Fambert-Eaton myasthenic syndrome; Fandau-Kleffner syndrome; lateral medullary (Wallenberg) syndrome; learning disabilities; Feigh's disease; Fennox-Gastaut syndrome; Fesch-Nyhan syndrome; leukodystrophy; Fewy body dementia; lissencephaly; locked-in syndrome; Fou Gehrig's disease (aka motor neuron disease or amyotrophic lateral sclerosis); lumbar disc disease; lyme disease-neurological sequelae; Machado-Joseph disease; macrencephaly; megalencephaly; Melkersson-Rosenthal syndrome; Menieres disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In one embodiment, the PGM is used to treat a “neurodegenerative diseases or disorder,” which refers to a type of neurological disease or disorder marked by the loss of nerve cells, including, but not limited to, Alzheimer's disease, Parkinson's disease, amyotrophic lateral sclerosis, tauopathies (including frontotemporal dementia), and Huntington's disease. In some embodiments, a neurodegenerative disease or disorder is Alzheimer's disease. Causes of Alzheimer's disease are poorly understood but in the majority of cases are thought to include a genetic basis. The disease is characterized by loss of neurons and synapses in the cerebral cortex, resulting in atrophy of the affected regions. Biochemically, Alzheimer's is characterized as a protein misfolding disease caused by plaque accumulation of abnormally folded amyloid beta protein and tau protein in the brain. Symptoms of Alzheimer's disease include, but are not limited to, difficulty remembering recent events, problems with language, disorientation, mood swings, loss of motivation, self-neglect, and behavioral issues. Ultimately, bodily functions are gradually lost, and Alzheimer's disease eventually leads to death. Treatment is currently aimed at treating cognitive problems caused by the disease (e.g., with acetylcholinesterase inhibitors or NMDA receptor antagonists), psychosocial interventions (e.g., behavior-oriented or cognition-oriented approaches), and general caregiving. There are no treatments currently available to stop or reverse the progression of the disease completely.

In one embodiment, the PGM is used to treat a “proliferative disease or disorder,” which refers to a disease or disorder that occurs due to abnormal growth or extension by the multiplication of cells (Walker, Cambridge Dictionary of Biology, Cambridge University Press: Cambridge, UK, 1990). A proliferative disease or disorder may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

### Target Gene

The target gene used in the present disclosure is not particularly limited as long as it is a gene that produces and expresses RNA (mRNA, lncRNA, miRNA, etc.) in vitro or in a cell (preferably wherein the cell is a prokaryotic cell or eukaryotic cell, preferably a mammalian cell, a cell of a non-human primate, or a human cell). In one embodiment, the target gene encodes a protein. In one embodiment, the target gene encodes a microRNA. In one embodiment, the target gene encodes a long noncoding RNA. The target gene may be selected from among any gene whose increased or decreased expression is beneficial in the treatment of the selected physiological or pathological condition of disease, disorder, cellular stress, or injury, examples of which are described below.

In one embodiment, there is an endogenous TFBS in the target gene, where the TF may, due to proximity, translocate from the PGM to the endogenous binding site. In one embodiment, the TF bound to the PGM may stay bound to the PGM irrespective of the presence of a binding site in the gene. Because the action of TFs may depend on their general proximity to the transcription start site or the chromatin associated with the gene, the co-linearity of the bound DNA with the gene is not required.

In one embodiment, the target gene is a gene that has a binding site for the TF that is brought in via the PGM. In this case, the TF may be known to modulate expression of that gene. In some embodiments, the presence of a TFBS in the target gene for the TF in the PGM is identified first with the methods of the disclosure.

In another embodiment, the target gene does not have a known binding site for the TF that is brought in via the PGM. Instead, the PGM brings in the TF in proximity to the promoter of the target gene via the DNA binding module and that is sufficient for the TF to enhance or decrease expression of the target gene. In other words, via the PGM, the target gene may be controlled by a TF that otherwise does not regulate expression of the target gene without the PGM. Thus, the PROTEGE platform extends to the modulation of the expression of any desirable/undesirable target gene via the PGM, because the PGM is designed to specifically bind the promoter of that target gene through the sgRNA/dCas portion, which then brings to that target gene a TF that is activated by a signal associated with the condition of interest (e.g., HIF-1alpha, activated by hypoxia) through the TFBS of the PGM.

Accordingly, the disclosure provides a method of very specifically regulating expression of a target gene in a cell-specific manner because only a cell exposed to the signal that activates the TF will have that TF brought into close proximity to the target gene via the PGM. In the absence of the signal, the PGM may be bound to the promoter region of the target gene, but nothing happens because there is no TF in the PGM. The TFBS is empty until the signal activates the TF, which then binds to the PGM and activates or reduces expression of the target gene.

In one embodiment, the target gene is selected from among the following categories: Fc Receptor, IgG-Fc control, cytokine, interleukin, growth factor, kinase, nuclease, protease, enzyme, stem cell protein, epigenetic protein, cancer protein, immunotherapy protein, CD molecule protein, receptor protein (e.g., cytokine, growth factor, B cell, monocyte, granulocyte, NK cell, Stem cell, T cell, and dendritic cell receptors), TNF superfamily, B7 family, TGFbeta family, cell therapy protein, immune checkpoint protein.

In one embodiment, the target gene is selected from among pro-inflammatory and anti-inflammatory genes. In one embodiment, such genes are selected from Cytokines (GM-CSF, IFN alpha, IFN gamma, IL-1 alpha, IL-1 beta, IL-4, IL-6, IL-8, IL-10, IL-12p70, IL-13, IL-17A (CTLA-8), and TNF alpha; Chemokines (IP-10 (CXCL10), MCP-1 (CCL2), MIP-1 alpha (CCL3), MIP-1 beta (CCL4); and/or Cell adhesion and inflammatory response genes (ICAM-1, CD62E (E-selectin), CD62P (P-Selectin). In one embodiment, the target gene encodes an anti-inflammatory cytokine. In one embodiment, the cytokine may be selected from IL-1 beta, IL-4, IL-6, IL-IRA, IL-4, IL-6, IL-10, IL-11, IL-13, and TGFbeta and it is desirable to upregulate its expression with a PGM. In one embodiment, the target gene encodes a pro-inflammatory cytokine and it is desirable to downregulate its expression with the PGM. In one embodiment, the pro-inflammatory cytokine is IL-1β, IL-6, and TNF-α.

In one embodiment, the target gene is selected from among receptors that relate to innate immunity. Table 3. The innate immunity receptors that recognize pathogens also have an important role in signaling for the induced responses responsible for local inflammation, the recruitment of new effector cells, the containment of local infection, and the initiation of an adaptive immune response. In one embodiment, the target gene is a co-stimulatory immune checkpoint target or a co-inhibitory immune checkpoint target, which may be useful in the treatment of cancer and respond to various cellular and extracellular signals. Table 4 and Table 5.

TABLE 4
Exemplary Target genes Related to Immunity

| | CO-STIMULATORY | CO-INHIBITORY |
|---|---|---|
| GENES RELATED TO | IMMUNE CHECKPOINT | IMMUNE CHECKPOINT |
| INATE IMMUNITY | TARGETS | TARGETS |
| TRA2A | CD155/PVR protein | PD1/PDCD1/CD279 |
| CD16a/Fc gamma RIIIa, | CD40/TNFRSF5 protein | protein |
| CD16b/Fc gamma RIIIb | OX40/CD134 protein | CTLA-4/CD152 protein |
| CD16-2/FCGR4 | HVEM/TNFRSF14 | B7-H3/CD276 protein |
| CD32a/FCGR2A/Fc gamma | protein | HVEM/TNFRSF14 |
| RIIA | CD28/TP44 protein | protein |
| CD32b/FCGR2B/Fc gamma | GITR/TNFRSF18 protein | Galectin-9/LGALS9 |
| RIIB | ICOS/AILIM/CD278 | SIRP alpha/CD172a |
| TRA2B | protein | protein |
| C5AR1/C5R1/CD88 | CD226/DNAM-1 protein | TIGIT/VSTM3 protein |
| G-CSF R/CD114/CSF3R | CD40L/CD154/ TNFSF5 | 2B4/CD244 protein |
| IL6R/IL-6R/CD126 | protein | PD-L1/B7-H1/CD274 |
| CD155/PVR | OX-40L/TNFSF4/CD252 | protein |
| KIR2DL4/CD158D | protein | CD80/B7-1 protein |
| CD180/RP105 | TNFSF14/LIGHT/ | B7-H4/B7S1/B7x protein |
| CCR2 | CD258 protein | CEACAM1/CD66a |
| CCR4/CD194 | CD80/B7-1 protein | protein |
| CCR5 | CD137/4-1BB protein | TIM-3/HAVCR2 protein |
| CCR8 | 4-1BBL/CD137L protein | CD155/PVR protein |
| IL13RA1/IL-13RA1 | CD27 protein | PD-L2/B7-DC/CD273 |
| IL18R1 | CD70/CD27L/TNFSF7 | protein |
| IL18RAP | protein | Indoleamine 2,3- |
| TLR1/CD281 | CD86/B7-2 protein | dioxygenase/IDO protein |
| TLR2 (CD282) | GITR Ligand/TNFSF18 | LAG3/CD223 protein |
| TLR3/CD283 | ICOS Ligand/B7-H2 | BTLA protein |
| TLR4/TLR-4 | protein | CD47 protein |
| TLR6 | CD155/PVR protein | CD86/B7-2 protein |
| TLR10 | CD40/TNFRSF5 protein | VISTA/B7-H5/GI24 |
| CSF2RA | OX40/CD134 protein | protein |
| CX3CR1 | HVEM/TNFRSF14 | CD160 protein |
| CXCR4/CD184 | protein | CD48/SLAMF2 protein |
| EGFR/HER1 | CD28/TP44 protein | Galectin-9/LGALS9 |
| FPR3 | GITR/TNFRSF18 protein | SIRP alpha/CD172a |
| IL-1RA/IL1RN | ICOS/AILIM/CD278 | protein |
| IL-1RAcP/IL-1R3 | protein | TIGIT/VSTM3 protein |
| TNFR1/TNFRSF1A/CD120a | CD226/DNAM-1 protein | 2B4/CD244 protein |
| TNFR2/TNFRSF1B/CD120b | CD40L/CD154/TNFSF5 | PD-L1/B7-H1/CD274 |
| TRAF3/CD40BP | protein | protein |
| TRAF3IP1 | OX-40L/TNFSF4/CD252 | CD80/B7-1 protein |
| | protein | B7-H4/B7S1/B7x protein |
| | TNFSF14/LIGHT/ | CEACAM1/CD66a |
| | CD258 protein | protein |
| | CD80/B7-1 protein | TIM-3/HAVCR2 protein |
| | CD137/4-1BB protein | CD155/PVR protein |
| | 4-1BBL/CD137L protein | |
| | CD27 protein | |
| | CD70/CD27L/TNFSF7 | |
| | protein | |
| | CD86/B7-2 protein | |
| | GITR Ligand/TNFSF18 | |

In one embodiment, expression of the target gene is helpful in the treatment of cancer. In one embodiment, the target gene is selected from those in Table 5.

TABLE 5
Exemplary Target Genes Whose Expression Is Useful
for Cancer Therapy and Their Therapeutic Actions

| Cytokines | Therapeutic actions |
|---|---|
| IL-2 | Enhances NK cell and CD8 T cell function; |
| | increases vascular permeabilty |
| IL-3 | Enhances cancer antigen presentation |
| IL-4 | Enhances eosinophil function and T-cell |
| | activation |
| IL-6 | Enhances T-cell and B-cell function; inhibition |
| | of IL-6 reduces lymphoproliferation |
| IL-7 | Enhances T-cell function |
| IL-10 | Inhibits cancer antigen presentation |
| IL-12 | Enhances Th1 immunity and cytotoxicity; |
| | anhibits angiogensis |
| IL-13 | Inhibits cytotoxicity against viral neoplasms |
| IL-15 | Enhances cytotoxicity |
| IL-18 | Enhances Thl immunity and cytotoxicity; |
| | inbibits angiogenesis |
| M-CSF | Enhances macrophage function |
| GM-CSF | Enhances cancer antigen presentation |
| IFN-α | Enhances cancer antigen presentation and |
| | cytotoxicity |
| IFN-γ | Enhances cancer antigen presentation and |
| | cytotoxicity |
| TNF-α | Induces tumor-cell apoptosis; activates |
| | endothelium and granulocytes |
| TRAIL | Induces tumor-cell apoptosis |
| FLT3 ligand | Stimulates dendritic-cell and NK-cell function |
| Lymphotactin | Enhances T-cell recruitment |
| TGF-β | Inhibits T-cell effector function |

In one embodiment, the target gene is a cytokine that plays a role in asthma. Asthma differs from other chronic inflammatory disorders, such as rheumatoid arthritis, Crohn's disease and psoriasis, in exhibiting a characteristic cytokine response dominated by Th2 cytokines, the majority of which are encoded in a small cluster on chromosome 5q32-34. It has been suggested that this coordinated regulation of the immune response in favor of Th2 cytokines, which include interleukin (IL)-3, IL-4, IL-5, IL-6, IL-9, IL-13 and granulocyte-macrophage colony stimulating factor (GM-CSF), results from a reduction in the inhibitory influence of Th1 cytokines, especially IL-18, IL-12 and interferon-γ, and, as a consequence, results in Th2 polarization of the immune response by default. This imbalance between Th1- and Th2-type immunity in those destined to become atopic manifests early in life, and possibly prenatally.

In one embodiment, the target gene is a gene involved in rheumatoid arthritis. Rheumatoid arthritis (RA) is a chronic systemic inflammatory disease that is characterized by persistent intense immunological activity, local destruction of bone and cartilage, and a variety of systemic manifestations. CD4 T cells play a central role in initiating and perpetuating the chronic autoimmune response characteristic of rheumatoid inflammation. In one embodiment, the target gene is IL-4, IFN-gamma, IL-10, or a Th1/Th2 cytokine.

In one embodiment, the target gene is involved in sepsis. In one embodiment, the target gene is selected from an IL-1 family member, an IL-1 receptor family member, a member of the TNF family, a member of the TNF Receptor family, an Interferon, an IFN receptor, a member of the IL-6, IL-10, IL-6 receptor and IL-10 receptor family, a member of the TGF beta or TGF beta receptor family, a chemokine, and a chemokine receptor.

In one embodiment, the target gene is a tumor suppressor gene and expression of the gene is advantageous, in which case the PGM is designed to enhance its expression. In one embodiment, the target gene is a mutated tumor suppressor gene whose expression is disadvantageous, in which case the PGM is designed to inhibit its expression. The human genome encodes over 2000 different TFs, many of which are expressed in a cell type-specific manner to coordinate gene expression programs underlying a vast array of cellular processes (see, e.g., Lee T I, Young R A. Transcriptional regulation and its misregulation in disease. Cell. 2013; 152:1237-1251). In one embodiment, the target gene is a pro-apoptotic gene and although expression of some other apoptotic genes is triggered by an extracellular signal (e.g., glucocorticoids) it is desirable to express additional pro-apoptotic genes in the cell in response to the signal. In this case, the PGM is designed to bind to a TF that responds to glucocorticoids and the PGM TF is brought into close proximity to a desired pro-apoptotic gene via the sgCNA. In one embodiment, the glucocorticoid will normally trigger expression of the proapoptotic BIM gene (BCL2 interacting mediator of cell death) in cancer cells, but the PGM brings the glucocorticoid-responsive TF into close proximity to one or more additional pro-apoptotic target genes, whose expression is also beneficial but would not be activated in the absence of the PGM. Examples of tumor suppressor genes include TP53, MYC. Examples of pro-apoptotic genes (i.e., proteins) include caspases, the amyloid-B peptide, some members of the Bcl-2 family of proteins, the p53 gene, BAX, BAK, BCLX, BAD, BID BIK, HRK, and the heat shock proteins. Examples of anti-apoptotic genes include BCL-2, BCL-XL, BCL-W, BFL-M, BRAG-1, MCL-1 and A1/BFL-1.

In some embodiments, the target gene is an enzyme. In some embodiments, the enzyme is selected from enzymes having one of more of the functions described in Table 5.

TABLE 5
Target Genes that are Enzymes and
Their Classification by Function

| | |
|---|---|
| tRNA synthetase | AARSD1 |
| | ART1 |
| | CARS |
| | DARS |
| | DARS2 |
| | DUS1L |
| | FARS2 |
| | FARSB |
| | HARS |
| | KARS |
| | NARS2 |
| | QARS |
| | RARS |
| | SARS |
| | SARS2 |
| | TARS |
| | TYW3 |
| | WARS |
| | WARS2 |
| | YARS |
| | YARS2 |
| Ubiquitin Modifiers | CENPU |
| | CNPY2 |
| | CUEDC2 |
| | HGS |
| | RNF11 |
| | RPS27A JOSD1 |
| | OTUB1 |
| | OTUB2 |
| | UBXN6 |
| | UCHL1 |
| | UCHL3 |
| | UCHL5 |
| | USP5 |
| | USP7 |
| | USP8 |
| | USP10 |
| | USP18 |
| | USP21 |
| | USP22 |
| | USP29 |
| | USP30 |
| | USP32 |
| | USP33 |
| | USP37 |
| | USP38 |
| | USP46 |
| | USP49 |
| Carbonic Anhydrase | Carbonic Anhydrase I/CA1 |
| | Carbonic Anhydrase II/CA2 |
| | Carbonic Anhydrase III/CA3 |
| | Carbonic Anhydrase IV/CA4 |
| | Carbonic Anhydrase VA/CA5A |
| | Carbonic Anhydrase VB/CA5B |
| | Carbonic Anhydrase VII/CA7 |
| | Carbonic Anhydrase VIII/CA8 |
| | Carbonic Anhydrase IX/CA9 |
| | Carbonic Anhydrase X/CA10 |
| | Carbonic Anhydrase XI/CA11 |
| | Carbonic Anhydrase XII/CA12 |
| | Carbonic Anhydrase XIII/CA13 |
| | Carbonic Anhydrase XIV/CA1 |
| Oxidative Stress Enzymes | Prooxidant Enzymes |
| | AOX1/Aldehyde Oxidase |
| | Syk |
| | VAP1/AOC3 |
| | NOXA1 |
| | NOX4 |
| | Antioxidant Enzymes |
| | GPX1 |
| | GPX2 |
| | GPX3 |
| | GPX7 |
| | SOD1 |
| | SOD2 |
| | Redox Enzymes |
| | AOC3 |
| | EGLN3 |
| | GFER |
| | HAO1 |
| | HPGDS |
| | P4HB |
| | TPH1 |
| | TPH2 |
| | Others |
| | BLVRB |
| | CAT |
| | DBH |
| | GLO1 |
| | GLRX2 |
| | GST |
| | GSTM2 |
| | GSTP1 |
| | GSTZ1 |
| | HAGH |
| | MAOA |
| | PIANP |
| | PON1 |
| | PON2 |
| | PON3 |
| | PRDX1 |
| | PRDX2 |
| | PRDX3 |
| | PRDX4 |
| | PRDX5 |
| | PRDX6 |
| | PRKCQ |
| | TXN |
| | TXN2 |
| | TXNRD1 |
| | TXNDC17 |
| Lipid Metabolism Enzymes | Pyrophosphatase (ENPP) |
| | Carboxylesterase (CES) |
| | Sphingosine Kinase (DPHK) |
| | Phospholipase |
| | AMACR |
| | ARSA |
| | ASAH2 |
| | FASN |
| | GK1 |
| | GK2 |
| | GK3P |
| | GK5 |
| | HAO1 |
| | HPGD |
| | LPL |
| | LTA4H |
| | MVK |
| | NAMPT |
| | PCSK9 |
| | CEL |
| | PLD1 |
| | PNPLA2 |
| | PNPLA3 |
| | PON1 |
| | PON2 |
| | PON3 |
| | SGPL1 |
| | SMPD1 |
| | SMPD3 |
| Ubiqutin-like Modifiers | |
| Histone Modifying Enzyme | |
| Methyltransferases and Demethylases | |
| Phosphatases and regulators | |
| Hydrolases | |
| Oxidoreductases | |
| Transferases | |

Exemplary PGMs and their Application

### Wound Healing

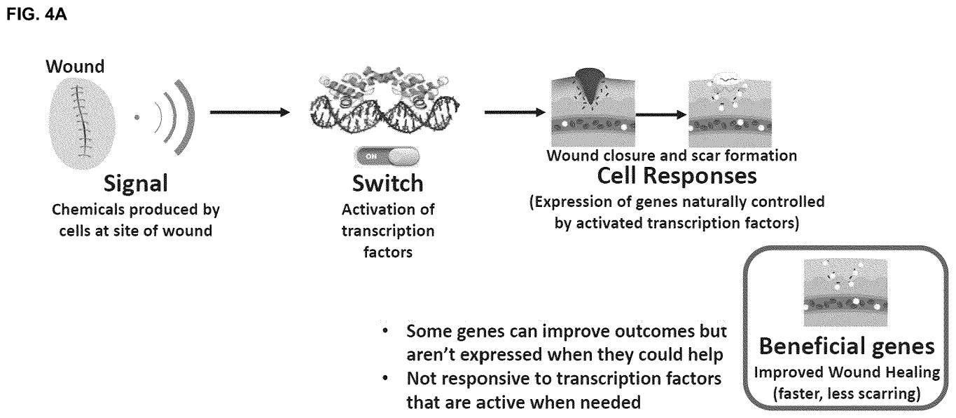

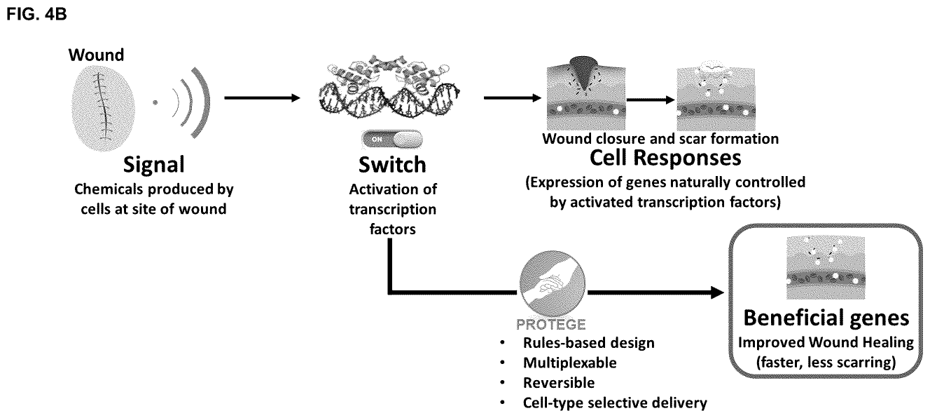

In one embodiment, the PGM is designed to correct the imbalance between TGF-β1 and TGF-B3 in wounds, which slows wound healing and causes scarring. FIGS. 4A and 4B. In one embodiment, the transcription factor FOXO1 or SMAD may be activated by an inflammatory signal and then brought to the promoter of the TGF-β3 gene via the PROTEGE platform to promote its expression and accelerate wound healing with reduced scarring. In one embodiment, this PGM is delivered topically to fibroblasts.

### Radiation Exposure

In one embodiment, the PGM is designed to reduce side effects of radiation exposure. In one embodiment, the PGM targets the GCSF gene, whose expression promotes hematopoiesis and mobilization of hematopoietic stem cells. In one embodiment, the PGM has a TFBS for NF-kB or Nrf-2 transcription factors. These may be activated via free radicals generated during radiation exposure and brought into contact with the promoter region of the GCSF gene to promote its expression via the PGM, thereby reducing the side effects of radiation exposure. In one embodiment, the PGM is delivered intravenously to bone marrow adipocytes.

### Viral Infection

In one embodiment, the PGM is designed to treat a viral infection. In one embodiment, the PGM targets the IFN gene, whose expression suppresses viral replication. In one embodiment, the PGM has a TFBS for NF-kB, which may be activated in the presence of viral RNA and then brought into contact with the promoter of the IFN gene by the PGM to promote its expression, thereby treating viral infection. In one embodiment, the PGM is delivered intranasally/inhalation to the respiratory endothelium. In one embodiment, the PGM is delivered intravenously.

### Diabetic Nephropathy

In one embodiment, the PGM is designed to treat diabetic nephropathy. In one embodiment, the PGM targets the Klotho gene, whose expression can suppress Renal Fibrosis. In one embodiment, the PGM has a TFBS for USF2, which may be activated by high glucose levels and then brought into contact with the promoter of the Klotho gene by the PGM to promote its expression, thereby suppressing renal fibrosis. In one embodiment, the PGM is delivered to glomerular endothelial and/or mesangial cells intravenously.

### Atherosclerosis

In one embodiment, the PGM is designed to treat atherosclerosis. In one embodiment, the PGM targets the FGF-21 gene and/or the Klotho gene, whose expression decreases inflammatory and oxidative stress. In one embodiment, the PGM has one or more TFBS for NFAT, EGR1, STAT3, SREBP, and/or Nrf2, which may be activated in the presence of oxidized phospholipids and then brought into contact with the promoter of the FGF-21 gene and/or the Klotho genes by the PGM to promote their expression, thereby decreasing inflammatory and oxidative stress. In one embodiment, the PGM is delivered to the coronary artery endothelium intravenously.

### Cystic Fibrosis

In one embodiment, the PGM is designed to treat cystic fibrosis. In one embodiment, the PGM targets the HNF-3β and/or CaCC genes, whose expression decreases mucin levels (HNF-3β), balance Cl⁻/Na⁺ levels, and water accumulation (CaCC). In one embodiment, the PGM has a TFBS for the NF-kB gene, which may be activated by mucosal buildup and then brought into contact with the promoter of the HNF-3β and/or CaCC genes by the PGM to promote their expression, thereby decreasing mucin levels (HNF-3β), balance Cl−/Na+ levels, and water accumulation (CaCC). In one embodiment, the PGM is delivered intranasally/inhalation to the airway mucosal epithelium.

### Alzheimer's Disease

In one embodiment, the PGM is designed to treat Alzheimer's Disease. In one embodiment, the PGM targets NDBF and/or NGF genes, whose expression promotes neurite survival. In one embodiment, the PGM has a TFBS for NFAT, which may be activated by the presence of high Ca2+ levels, and then brought into contact with the promoter of NDBF and/or NGF genes by the PGM to promote their expression, thereby promoting neurite survival. In one embodiment, the PGM is delivered to entorhinal neurons via intrathecal administration.

### Oxidative Stress and Inflammation

In one embodiment, the PGM is designed to treat oxidative stress and/or inflammation. In one embodiment, the PGM targets the klotho gene, which encodes a membrane-bound and circulating protein that suppresses oxidative stress and inflammation. In one embodiment, this PGM comprises a TFBS for Nrf2, which may be activated by oxidative stress and/or inflammatory signals (may be mimicked by the addition of tert-butylhydroquinone (tBHQ)) and then brought into contact with the promoter of the klotho gene by the PGM to promote its expression, thereby suppressing oxidative stress and inflammation. In one embodiment, the PGM is delivered intranasally/inhalation to the airway mucosal epithelium. In one embodiment, the PGM is delivered to the coronary artery endothelium intravenously. In one embodiment, the PGM is delivered intranasally/inhalation to the respiratory endothelium. In one embodiment, the PGM is delivered intravenously.

### Cancer

In one embodiment, the PGM is designed to treat cancer. In one embodiment, the PGM targets a BH3-only gene, which encodes a protein that promotes apoptosis in tumor cells. In one embodiment, the PGM comprises a TFBS for one or more of HIF-1, p73, Sp1 or Fox03a, which may be activated by hypoxia, low pH, and/or high levels of lactic acid in the tumor microenvironment and brought into contact with the promoter of a BH3-only gene by the PGM, thereby triggering apoptosis in the tumor cells.

In one embodiment, the PGM targets one or more genes encoding glycolytic enzymes such as an hexokinase or a phosphoglycerate kinase, which stimulate glucose uptake by regulating glucose transporters GLUT1 and GLUT3. In one embodiment, this PGM comprises a TFBS for a TF that responds to glucose levels.

In one embodiment, target gene expression is increased by at least 1.5-fold, at least 2.0 fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0 fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 100-fold, at least 200-fold, at least 500-fold, or at least 1000-fold, relative to the gene expression level in the absence of the PGMs.

In one embodiment, target gene expression is decreased by at least 1.5-fold, at least 2.0 fold, at least 2.5-fold, at least 3.0-fold, at least 3.5-fold, at least 4.0-fold, at least 4.5-fold, at least 5.0-fold, at least 5.5-fold, at least 6.0-fold, at least 6.5-fold, at least 7.0-fold, at least 7.5-fold, at least 8.0-fold, at least 8.5-fold, at least 9.0-fold, at least 9.5-fold, at least 10.0 fold, at least 11-fold, at least 12-fold, at least 13-fold, at least 14-fold, at least 15-fold, at least 16-fold, at least 17-fold, at least 18-fold, at least 19-fold, at least 20-fold, at least 21-fold, at least 22-fold, at least 23-fold, at least 24-fold, at least 25-fold, at least 26-fold, at least 27-fold, at least 28-fold, at least 29-fold, at least 30-fold, at least 31-fold, at least 32-fold, at least 33-fold, at least 34-fold, at least 35-fold, at least 36-fold, at least 37-fold, at least 38-fold, at least 39-fold, at least 40-fold, at least 41-fold, at least 42-fold, at least 43-fold, at least 44-fold, at least 45-fold, at least 46-fold, at least 47-fold, at least 48-fold, at least 49-fold, at least 50-fold, at least 51-fold, at least 52-fold, at least 53-fold, at least 54-fold, at least 55-fold, at least 56-fold, at least 57-fold, at least 58-fold, at least 59-fold, at least 60-fold, at least 61-fold, at least 62-fold, at least 63-fold, at least 64-fold, at least 65-fold, at least 66-fold, at least 67-fold, at least 68-fold, at least 69-fold, at least 70-fold, at least 71-fold, at least 72-fold, at least 73-fold, at least 74-fold, at least 75-fold, at least 100-fold, at least 200-fold, at least 500-fold, or at least 1000-fold, relative to the gene expression level in the absence of the PGMs.

### Nucleic Acids and Vectors

In one embodiment, the disclosure provides nucleic acids that comprise one or more components of the PGMs disclosed herein. In one embodiment, the nucleic acid comprises one or more of: cRNA and/or cRNA module, a tracrRNA and/or a tracrRNA module, and an sgCNA, and/or a nucleic acid with a transcription factor binding site or a Transcription Factor Binding site module.

In some embodiments, the transcription factor binding site comprises one or more modifications, relative to the native sequence. In some embodiments, the one or more modifications comprises one or more transitions, one or more transversions, one or more insertions, one or more deletions, one more inversions, or any combination thereof. In one embodiment, the one or more transitions are selected from the group consisting of: (a) T to C; (b) A to G; (c) C to T; and (d) G to A. In one embodiment, the one or more transversions are selected from the group consisting of: (a) T to A; (b) T to G; (c) C to G; (d) C to A; (e) A to T; (f) A to C; (g) G to C; and (h) G to T. In one embodiment, the crRNA carries one or more modifications relative to the crRNA that hybridizes in its full extent to the target gene. In one embodiment, the one or more modifications comprises changing the native DNA gene sequence encoding the DNA binding protein (e.g., dCas) so that at least one of the following changes are introduced (1) a G:C basepair to a T:A basepair, (2) a G:C basepair to an A:T basepair, (3) a G:C basepair to a C:G basepair, (4) a T:A basepair to a G:C basepair, (5) a T:A basepair to an A:T basepair, (6) a T:A basepair to a C:G basepair, (7) a C:G basepair to a G:C basepair, (8) a C:G basepair to a T:A basepair, (9) a C:G basepair to an A:T basepair, (10) an A:T basepair to a T:A basepair, (11) an A:T basepair to a G:C basepair, or (12) an A:T basepair to a C:G basepair. In one embodiment, the one or more modifications comprises an insertion or deletion of 1, 2, 3, 4, 5, 6, 7, 8, 9, 10, 11, 12, 13, 14, 15, 16, 17, 18, 19, 20, 21, 22, 23, 24, or 25 nucleotides, optionally wherein the one or more edits comprises an insertion or deletion of 1-15 nucleotides. In some embodiments, the transcription factor binding site is at least 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity or homology relative to the native sequence. In one embodiment, the nucleic acid contains one or more chemically modified or non-natural nucleotides. In some embodiments, the inclusion of chemically modified or non-natural nucleotides increases the functional lifetime of the PGM in the cell.

In some embodiments, the disclosure provides a vector that comprises one or more nucleic acids of the disclosure. In some embodiments, the vector comprises a nucleic acid encoding the DNA binding protein of the disclosure (dCas). In some embodiments, the vector is a retroviral vector, a DNA vector, an RNA vector, an adenoviral vector, a baculoviral vector, an Epstein Barr viral vector, a papovaviral vector, a vaccinia viral vector, a herpes simplex viral vector, an adenovirus associated vector, a lentiviral vector, or any combination thereof

### Compositions

In one embodiment, the disclosure provides compositions comprising or consisting of one or more nucleic acids and/or proteins of the disclosure. In one embodiment, the disclosure provides compositions comprising one or more cells of the disclosure (preferably wherein the cell is a prokaryotic cell or eukaryotic cell, preferably a mammalian cell, a cell of a non-human primate, or a human cell). In some embodiments, the compositions are pharmacological compositions. In some embodiments, the compositions comprise or consist of one or more components of the PGMs described herein and are capable of being administered to a cell, tissue, or organism by any suitable means, such as by gene therapy, mRNA delivery, virus-like particle delivery, or ribonucleoprotein (RNP) delivery, and combinations thereof, as described above.

In one embodiment, the disclosure provides compositions for delivering the nucleic acids of the disclosure to a cell. In one embodiment, the compositions comprise or consist of a RNA, DNA, and/or protein component of the PGMs of the disclosure. In one embodiment, the compositions comprise or consisting of an entire PGM of the disclosure. In one embodiment, the compositions comprise a cRNA and/or cRNA module, a tracrRNA and/or a tracrRNA module, a Cas/DNA binding protein, an sgCNA, and/or a nucleic acid with a transcription factor binding site or a Transcription Factor Binding site module. More compositions are described above in the methods of delivery of the gene PGM system.

In one embodiment, the compositions comprise one or more cells comprising a crRNA and/or cRNA module, a tracrRNA and/or a tracrRNA module, a Cas/DNA binding protein, an sgCNA, a nucleic acid with a transcription factor binding site or a Transcription Factor Binding site module, and/or a PGM. In some embodiments, the composition further comprises a chemical that serves as a signal for activation/upregulation/inhibition/downregulation of the PGM-mediated gene expression. In some embodiments, the chemical is a drug.

In some embodiments, the compositions are pharmaceutical compositions. In some embodiments, the pharmaceutical composition comprises any of the compositions disclosed herein. In some embodiments, the pharmaceutical composition comprises any of the compositions disclosed herein and a pharmaceutically acceptable carrier. In some embodiments, the pharmaceutical composition comprises or consists of any of the polynucleotides and/or proteins disclosed herein and a pharmaceutically acceptable carrier. Any reference to a composition of the disclosure as “comprising” something, is also a reference to the same composition as “consisting of” that something, and also a reference to the same composition as “consisting essentially of” that something, even if not explicitly disclosed or enumerated herein.

Some examples of materials which may serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C2-C12 alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

### Kits

In one embodiment, the disclosure provides a kit. In one embodiment, the kit comprises one or more of the nucleic acids and/or vectors of the disclosure. In one embodiment, the kit further comprises a DNA-binding protein (e.g., dCas). In one embodiment, the kit comprises instructions for use. In one embodiment, the kit comprises components for preparing a pharmaceutical composition with the nucleic acids and/or cells of the disclosure.

### Alternative PGMs

The disclosure also provides modifications to the above embodiments, where the DNA binding molecules do not encompass a Cas protein. Accordingly, in one embodiment, any moiety with sufficient DNA binding specificity to address a single site in the target genome (e.g., human genome) may be used as the DNA binding module of a PGM. In one embodiment, the DNA binding molecules are arranged in tandem arrays of approximately six zinc finger motif units that bind to a chosen, unique site in the human genome. In one embodiment, such zinc finger arrays may have previously been conjugated with DNA modifying molecules such as nucleases and transcription factors to target those DNA modifying activities to the DNA proximal to the zinc finger recognition site. In one embodiment, the PGM comprises conjugation of nucleic acids to peptides such as zinc finger arrays and such methods may be used to append a transcription factor binding module comprising nucleic acid to the zinc finger array DNA binding module to create a PGM. As one example, synthesis of a zinc finger array by solid phase peptide synthesis allows incorporation of a dibenzocyclooctyne (DBCO) group by standard peptide coupling procedures to the amino terminus of the zinc finger peptide. A transcription factor binding module composed of nucleic acid bearing an azide group linked to its 3′ or 5′ terminus may be coupled with this terminal group by means of the well-known strain-promoted azide-alkyne cycloaddition reaction. Nucleic acids bearing an azide group may be readily prepared by reaction of an azide-bearing linker such as azidobutyrate NHS ester to an amino linker on an oligonucleotide synthesized by solid phased phosphoramidite chemistry.

In one embodiment, the DNA binding module of the PGM comprises TAL (transcription activator-like) effector proteins. Correspondence between the polypeptide sequence of TAL effectors and their DNA recognition sequence enable embodiments of proteins that bind to desired, unique DNA sequences. Any of the methods known to those with skill in the field to conjugate proteins to nucleic acids can be used to attach a transcription factor binding module comprising nucleic acids to a TAL effector DNA binding module to create a PGM. Such methods include but are not limited to attachment of a dibenzocyclooctyne (DBCO) group to the protein using any of a variety of crosslinking agents followed by coupling a nucleic acid module bearing an azide group by means of azide-alkyne cycloaddition or coupling a maleimide group appended to the nucleic acid module to the polypeptide through a cysteine by means of a Michael addition. In one embodiment, the TAL effector protein may be engineered to comprise two different DNA binding domains, one that binds the target DNA sequence of the PGM DNA binding module and another that binds to a duplex DNA component of the transcription factor binding module.

In addition to using a DNA oligonucleotide that folds to contain a known duplex DNA binding site of an endogenous transcription factor, a transcription factor binding module may be derived from a DNA or RNA aptamer that binds the desired transcription factor. Accordingly, in one embodiment, DNA and RNA aptamers may be generated to bind to a wide range of molecules, including proteins, including transcription factors, using methods known to those knowledgeable in the field. In one embodiment, a PGM with a transcription factor binding module comprising a DNA aptamer may be attached to a genomic DNA binding module to create a PGM by the same methodology and chemistry as a DNA hairpin, using for example DNA ligase. In that case, the aptamer may be synthesized with complementary sequences near the 3′ and 5′ ends of the DNA to promote formation of a duplex region with an overhang to allow ligation to cr and tracr components of the sgCNA. In the case of an RNA aptamer, the entire guide nucleic acid may be created by transcription of a DNA template.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments/aspects have been illustrated and described, it will be appreciated that various changes can be made without departing from the spirit and scope of the disclosure.

Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art. Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the embodiments explicitly disclosed above, below, and in the claims.

### EXAMPLES

### Example 1

### Preparation of Chimeric Guide Nucleic Acid

Cr module, tracr module, and transcription factor binding module oligonucleotides were obtained, fully deprotected and gel purified, from commercial providers.

Cr module:
(SEQ ID NO: 64)
5′CGGAGGCAGUCCCGGCUCGCGUUUUAGAGCUAdAdCdCdCTdGdAdCT
TdGdAdCdGT3′;
tracr module:
(SEQ ID NO: 65)
5′dAdAdGTdCdAdGdGdGTUAGCAAGUUAAAAUAAGGCUAGUCCGUUAU
CAACUUGAAAAAGUGGCACCGAGUCGGUGCUUUU3′;
transcription factor binding module:
(SEQ ID NO: 66)
5′dATdGdAdCTdCdAdGdCdAdCdAdATdGdGdCdGdAdAdAdGdCdCd
ATTdGTdGdCTdGdAdGTdCdATdAdCdGTdC3′;

The Cr module targets the sequence 5′CGGAGGCAGUCCCGGCUCGC3′ (SEQ ID NO: 67) in the promoter region of the klotho gene. This site was identified and selected with the target site selection tool CRISPick (https://portals.broadinstitute.org/gppx/crispick/public) using the CRISPRa function.

The transcription factor binding module is designed to fold into a hairpin structure containing a Nrf2 response element: 5′-ATGACTCAGCA-3′ (SEQ ID NO: 68).

The tracr module and transcription factor binding module were each obtained with a 5′ phosphate. Tracr module (7 nmoles) and Cr module (8 nmoles) were annealed in a total volume of 15 μL by warming to 65° C. for 1 minute followed by cooling to room temperature over 60 minutes. Transcription factor binding module (8 nmole in 41 μL) was annealed by heating to 95° C. for 1 minute and cooling to room temperature over 75 minutes. The annealed oligonucleotide modules were mixed and ligated in an 80 μL reaction containing 1 mM ATP, 1× ligase buffer, and 16,000 units of T4 DNA ligase. Following ligation overnight at room temperature, the ligase was inactivated by warming to 65° C. for 15 minutes. The mixture was then phenol extracted and the aqueous layer was desalted by gel filtration, eluting with 400 μL of water. The nucleic acid was precipitated with ethanol and sodium acetate, dissolved in water, and desalted again by gel filtration

### Example 2

### Preparation of Programmable Gene Modulator

Chimeric guide nucleic acid (80 pmoles) in 10 μL of phosphate buffered saline was warmed to 37° C. for 30 minutes followed by cooling to room temperature over 30 minutes.

Chimeric guide nucleic acid sequence:
(SEQ ID NO: 69)
5′CGGAGGCAGUCCCGGCUCGCGUUUUAGAGCUAdAdCdCdCTdGdAdCT
TdGdAdCdGTdATdGdAdCTdCdAdGdCdAdCdAdATdGdGdCdGdAdAd
AdGdCdCdATTdGTdGdCTdGdAdGTdCdATdAdCdGTdCdAdAdGTdCd
AdGdGdGTUAGCAAGUUAAAAUAAGGCUAGUCCGUUAUCAACUUGAAAAA
GUGGCACCGAGUCGGUGCUUUU3′
Klotho/Nrf2 sgNA example sequence:
(SEQ ID NO: 4)
5′-CGGAGGCAGTCCCGGCTCGCGUUUUAGAGCUAdAdCdCdCdTdGdAd
CdTdTdGdAdCdGdTdAdTdGdAdCdTdCdAdGdCdAdCdAdAdTdGdGd
CdGdAdAdAdGdCdCdAdTdTdGdTdGdCdTdGdAdGdTdCdAdTdAdCd
GdTdCdAdAdGdTdCdAdGdGdGdTUAGCAAGUUAAAAUAAGGCUAGUCC
GUUAUCAACUUGAAAAAGUGGCACCCGAGUCGGUGCUUUU-3′

The annealed chimeric guide nucleic acid was mixed with 78 pmole of recombinant dCas9 having a nuclear localization sequence fused to both N- and C-termini (NLS-dCas9-NLS, Novateinbio, PR-137213B).

Nuclear localization signal sequence-
(SEQ ID NO: 70)
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD-nuclear localization signal
sequence

The mixture was incubated at room temperature for 15 minutes to form the programmable gene modulator (PGM).

### Example 3

### Ex Vivo Assay for Transcription Factor Recruitment to Target DNA

### Preparation of Test Wells

Biotin-labeled oligonucleotide duplexes containing the 20 bp target sequence from the promoter of the human Klotho gene or a control sequence in which the target sequence was scrambled were immobilized on high binding capacity streptavidin-coated 8-well strips. Sense and anti-sense strands of the duplexes were obtained from a commercial source fully deprotected and gel purified.

Sense-strand target DNA:
(SEQ ID NO: 71)
5′CCTCGGCGCCCCTGCCCCCGCCCCCAGTGCCAGGGCGGAGGCAGTCC
CGGCTCGCAGGTAATTATTGCCAGCGGAGCCCGCCGGGGAGCG3′
Anti-sense strand target DNA:
(SEQ ID NO: 72)
5′CGCTCCCCGGCGGGCTCCGCTGGCAATAATTACCTGCGAGCCGGGAC
TGCCTCCGCCCTGGCACTGGGGGCGGGGGCAGGGGCGCCGAGG-
Biotin3′
Sense-strand scrambled target DNA:
(SEQ ID NO: 73)
5′CCTCGGCGCCCCTGCCCCCGCCCCCAGTGCCAGGGGGACGCGCGGGC
ACCGCTTCAGGTAATTATTGCCAGCGGAGCCCGCCGGGGAGCG3′
Anti-sense strand scrambled target DNA:
(SEQ ID NO: 74)
5′CGCTCCCCGGCGGGCTCCGCTGGCAATAATTACCTGAAGCGGTGCCC
GCGCGTCCCCCTGGCACTGGGGGCGGGGGCAGGGGCGCCGAGG-
Biotin3′

The anti-sense strands were obtained labeled with biotin at their 3′-termini. Sense and anti-sense oligonucleotides were combined in tris buffered saline (TBS) at a concentration of 8 μM and annealed by heating to 95° C. for 5 minutes followed by cooling to room temperature over 60 minutes. The hybridized duplex was diluted two-fold with 5× concentrated TBS, and 100 μL of the resulting solution was added to each streptavidin-coated well, followed by incubation for 72 hours at room temperature. Each DNA-coated well was washed with tris buffered EDTA (TE) followed by washing with TBS.

### Preparation of Nuclear Extract.

HEK293 cells were grown to 50-70% confluence in 10 cm dishes and treated with 50 μM freshly prepared tert-butylhydroquinone (tBHQ) in phosphate buffered saline (PBS) with 30% DMSO for 24 hours to activate Nrf2. For controls, cells were treated with PBS/30% DMSO. Cells were scraped from the dish in PBS and centrifuged at 3,200 rpm for 5 minutes at 4° C. Cells were washed once with PBS and the pellet was gently resuspended in 100 μL cold hypotonic buffer solution (20 mM Tris-HCl pH 7.4, 500 mM NaCl, 3 mM MgCl2). After a 15-minute incubation on ice, 5 μL of 10% NP40 lysis buffer (Sigma Aldrich) was added, and cells were vigorously vortexed for 10 seconds. The homogenate was centrifuged for 10 minutes at 3,000 rpm at 4° C. The supernatant containing the cytoplasmic fraction was discarded. To the pellet containing the nuclear fraction, 50 μL Invitrogen Cell Extraction Buffer (Cat #FNN0011, Invitrogen) supplemented with 1 mM PMSF and protease inhibitor cocktail was added, followed by incubation for 30 minutes on ice with vortexing every 10 minutes. The solution was centrifuged for 30 minutes at 14,000×g at 4° C. and supernatant containing the nuclear fraction was transferred to a separate tube. Total protein was quantified by Bradford assay using (Pierce Micro-BCA Assay).

### Assay for Nrf2 Recruitment.

Nrf2 binding to PGM bound to target DNA was assessed using components from a Nrf2 transcription factor assay kit (Abcam, ab207223). Freshly prepared PGM was added to wells with oligonucleotide duplexes immobilized as described above, followed by incubation overnight at room temperature. Unbound PGM was removed by washing with TE. Nuclear extracts (20 μg of total protein) from HEK298 cells, treated with tBHQ or vehicle only, were added to the wells, followed by incubation at room temperature for one hour. Each well was washed 3× with 200 μL of the wash buffer provided by the assay kit. Rabbit anti-Nrf2 antibody provided with the kit (100 μL, 1:1000 dilution) was added followed by incubation for one hour at room temperature and washing 3× with 200 μL of the wash buffer provided by the assay kit. Anti-rabbit HRP antibody (100 μL, 1:1000 dilution) was added, followed by incubation at room temperature for one hour and washing 4× with 100 μL wash buffer. Developing solution was added (100 μL) and the wells were incubated for 10 minutes at room temperature prior to addition of Stop Solution (100 μL). Nrf2 binding to wells was quantified by absorbance at 450 nm compared to control wells developed without anti-Nrf2 antibody.

### Example 4

### Signal-Dependent Transcriptional Modulation in Cultured Cells

HEK293 cells were plated onto 6-well plates at 3×10⁵ cells per well in 2 mL of complete growth medium (DMEM with 10% FBS). Cells were transfected with the PGM described above when they had reached 30-50% confluence. PGM was freshly prepared as described above, and Opti-MEM medium (500 μL) was added to the PGM, followed by addition of 50 μL Cas9 Plus reagent (Invitrogen, CMAX00008). The resulting solution was added to a solution of 500 μL of Opti-MEM and 30 μL CRISPRMAX transfection reagent (Invitrogen, CMAX00008). The mixture was briefly vortexed and incubated at room temperature for 10 minutes. In control experiments lacking PGM, a mock PGM solution was prepared by replacing the chimeric guide nucleic acid with water and the NLS-dCas9-NLS with Tris-HCl. The PGM or mock PGM solution (250 μL) was added to the cells followed by incubation for 16 hours. tBHQ solution, freshly prepared as described above, or vehicle was added to each well and cells were incubated for an additional 24 hours.

Total RNA was isolated using the PureLink RNA Mini Kit and quantified spectrophotometrically. Total RNA from each sample (250 ng) was reverse transcribed by Thermo Scientific Verso cDNA synthesis kit using a 3:1 (volume:volume) mixture of random hexamers to anchored oligo-dT primers in a 30 μL reaction according to manufacturer's protocol. Each condition was assayed for the target gene, Klotho, and endogenous reference gene GAPDH using Taqman Gene Expression Assays (ThermoFisher assays Hs00934627_m1 and Hs02786624_g1 respectively) and Taqman Fast Advanced Master Mix (ThermoFisher). Hs02786624_g1 covers a 157 nt amplicon in GAPDH exon 7. Hs00934627_m1 covers a 108 nt amplicon spanning exons 2 and 3 in KL. Reactions contained 4 μL of two-fold diluted cDNA in 20 μL qPCR reactions in a 96-well plate. Data were collected using the Bio-Rad CFX96 Touch Real-Time PCR Detection System and analyzed using the ΔΔC_{q} method to calculate relative gene expression.

### Example 5

Association of a Physiologically Responsive Transcription Factor with a Target DNA Sequence Directed by a Programmable Gene Modulator (PGM)

As previously explained in FIG. 1A, the principle for a physiologically responsive gene expression modulator according to the disclosure is the following: a transcription factor (TF) is activated by a physiologic stimulus. Examples of physiologic stimuli include oxidative stress or growth factor signaling. The responsive gene expression modulator is a ribonucleoprotein complex composed of a disabled CRISPR-associated protein, dCas9, and a chimeric guide nucleic acid. The chimeric guide nucleic acid comprises a DNA hairpin that incorporates a binding site for the activated TF, a crRNA sequence, and a tracrRNA sequence. This complex binds to a sequence of genomic DNA proximal to a target gene that is to be made responsive to the physiologic signal. The binding site is programmed by the crRNA sequence in the guide nucleic acid. Association of the activated TF with the bound dCas9 complex brings the TF in proximity to the target gene resulting in modulation of target gene transcription.

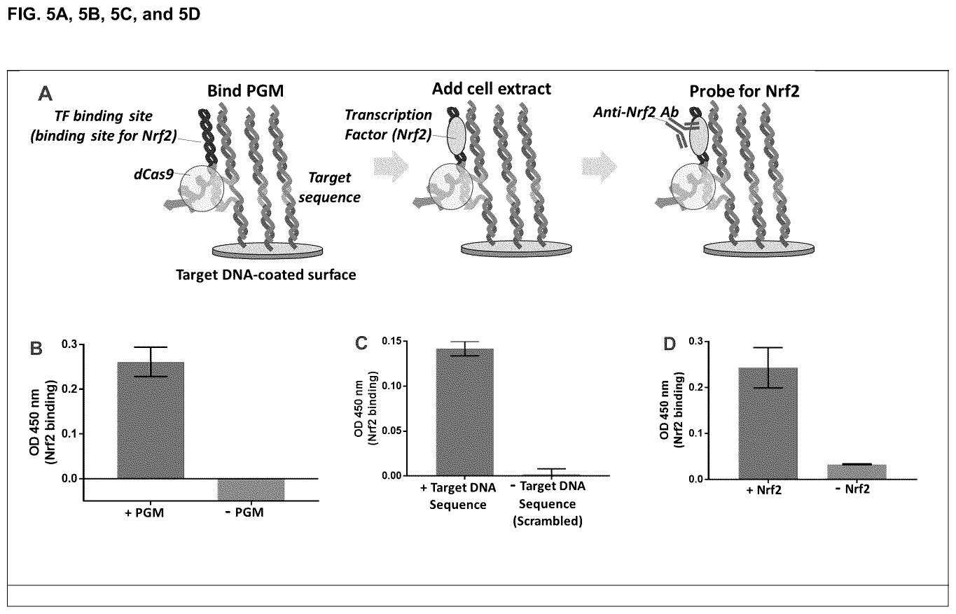

Here, a gene modulator comprising dCas9 and a DNA response element to transcription factor Nrf2 recruited activated Nrf2 to the DNA sequence targeted by the guide nucleic acid. FIG. 5A. The target DNA sequence is a 20-base pair sequence (pink and gold) contained within a DNA duplex immobilized in the well of a multi-well plate. The gene modulator comprises dCas9 (yellow circle) complexed with a single guide nucleic acid comprising a crRNA module (turquoise) complementary to the target sequence, a tracrRNA module (teal), and a DNA module that forms a hairpin structure incorporating the Nrf2 response element in its stem (red). Binding of the gene modulator to the immobilized target DNA is followed by addition of a nuclear extract from Hek293 cells that have been treated with tert-butylhydroquinone (tBHQ) to stimulate activation and nuclear localization of Nrf2. After washing the well to remove unbound Nrf2, bound Nrf2 is detected with an anti-Nrf2 antibody, visualized by optical absorbance at 450 nm after treatment with HRP-conjugated anti-rabbit secondary antibody and development with HRP substrate FIGS. 3B.-3D. Each value is the mean of three replicates in separate wells. Error bars are the standard deviation in the mean. FIG. 5B. Dependence of Nrf2 binding on presence of the PGM. For “-PGM” wells, PBS was added instead of PGM solution. FIG. 5C. Dependence of Nrf2 binding on presence of target DNA sequence immobilized in well. In the “-Target DNA Sequence” wells, the immobilized duplex contained a scrambled version (same sequence composition, different sequence) of the target sequence in place of the target sequence. FIG. 5D. Dependence of Nrf2 binding on Nrf2 activation. Nuclear extract added to “-Nrf2” wells was from cells untreated with tBHQ.

Nrf2 was activated by addition of tert-butylhydroquinone (tBHQ) to the cultured cells. tBHQ is a well-known activator of Nrf2. It has been shown to react with Keap1, a protein that localizes Nrf2 to the cytosol. Reaction of tBHQ with Keap1 promotes translocation of Nrf2 to the nucleus Li, W., and Kong, A. N. (2009). Molecular mechanisms of Nrf2-mediated antioxidant response. Mol. Carcinog. 48, 91-104.

Binding of the PGM to the target DNA followed by addition of a nuclear extract from cells in which Nrf2 has been activated resulted in association of Nrf2 with the target DNA. Nrf2 binding was not detected in the absence of the PGM. Nrf2 binding also depends on the correct sequence in the target DNA:Nrf2 did not bind to wells in which in which the DNA sequence targeted by the guide nucleic acid has been replaced with a scrambled sequence. Association of Nrf2 with the immobilized target DNA also depends on biochemical activation of Nrf2. Addition to the well of a nuclear extract from cells that have not been treated with tBHQ to activate Nrf2 did not result in binding of Nrf2.

### Example 6

### Transcriptional Modulation by a PGM of a Target Gene, Klotho, in Response to Biochemical Activation of an Endogenous Transcription Factor

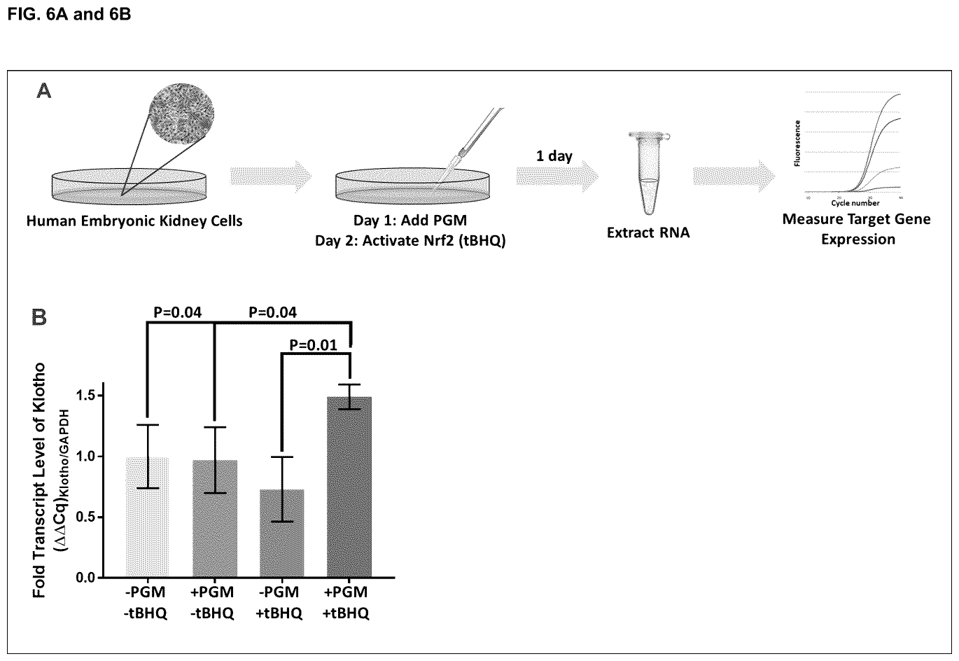

FIGS. 6A and 6B show Nrf2-dependent modulation of klotho transcription in cultured cells. Human embryonic kidney cells were treated with a PGM targeted to the klotho promoter and containing the Nrf2 response element. After 16 hours, Nrf2 was activated with tBHQ. Total RNA was isolated after an additional 24 hours, and klotho expression relative to GAPDH expression was measured by RT-qPCR. FIG. 6A. The target sequence of the PGM was a 20 base pair sequence upstream of the transcription start site of the gene for klotho, a membrane-bound and circulating protein that suppresses oxidative stress and inflammation. The transcription factor binding module of the PGM contained the Nrf2 response element. Addition of the PGM alone did not significantly affect transcription of klotho relative to transcription of the housekeeping gene, GAPDH, and treatment of the cultured cells with tBHQ caused a small decrease in klotho transcription. However, treatment with the PGM followed by activation of Nrf2 with tBHQ, resulted in a two-fold increase in klotho transcription compared with tBHQ treatment alone. FIG. 6B.

1. An engineered non-naturally occurring system comprising a programmable gene modulator (PGM) for reversibly modifying expression of a target gene of interest in a cell in response to one or more intracellular or extracellular environmental signal(s), or the sgCNA subcomponent thereof, comprising the following subcomponents:
an endonuclease-defective DNA-binding polypeptide; and
a chimeric nucleic acid (sgCNA) comprising a CRISPR RNA (crRNA), a trans-activating crRNA (tracrRNA), and at least one nucleic acid segment comprising at least one transcription factor binding site;
wherein the crRNA comprises a sequence complementary to a nucleic acid sequence in the promoter region of the target gene of interest and each transcription factor binding site(s) in the PGM bind(s) to at least one endogenous transcription factor that is activated in a cell comprising the PGM in response to the environmental signal(s) and then recognizes and binds to the transcription factor binding site of the PGM which is bound through the crRNA to the promoter of the gene of interest, thereby bringing the transcription factor into proximity with the gene of interest and activating or suppressing expression of the gene of interest in response to the environmental signal(s).

2. The engineered non-naturally occurring system of claim 1, or the sgCNA subcomponent thereof, wherein the system comprises at least one feature selected from the group consisting of:
(i) at least one transcription factor binding site in the PGM is also present in the target gene;
(ii) at least one transcription factor binding site in the PGM is not an endogenous transcription factor binding site in the target gene;
(iii) the PGM recruits the endogenous transcription factor(s) to the gene of interest when the endogenous transcription factor(s) has/have been activated in response to an environmental signal(s), thereby activating gene expression in response to the environmental signal(s) in a cell-specific manner;
(iv) the DNA-binding polypeptide is a nuclease-deficient cas polypeptide;
(v) the DNA-binding polypeptide is a dCas polypeptide;
(vi) the DNA-binding polypeptide is an endonuclease-defective sequence-specific DNA binding protein fused with at least one copy of a nuclear localization signal;
(vii) the transcription factor binding sequence comprises one or more sequences selected from: SEQ ID NO: 1, SEQ ID NO: 2, SEQ ID NO: 3, SEQ ID NO: 5, 5′-CACGTG-3′, 5′-TGA(G/C)TCA-3′, 5′-GGGNNNNNCC-3′, 5′-TGACGTCA-3, 5′-(C/T)AAC(G/T)G-3′, 5′-TTNCNNNAA-3′, 5′-GGA(A/T)-3′, 5′-GTCTAGAC-3′, 5′-TT(G/A)TTTAC-3′, 5′-CACGTG-3′, 5′-GCGTGGGCG-3′, 5′-TTCCCGGAA-3′, 5′-TCACNCCAC-3′ and 5′-(A/T)GGAAAN(A/T/C)N-3′ wherein N is any base; and
(viii) the tracrRNA binds dCas9.

3. (canceled)

4. The engineered non-naturally occurring system of claim 1, or the sgCNA subcomponent thereof, wherein the transcription factor(s) is/are at least one selected from the group consisting of a transcription factor identified as a transcription factor that is known to be activated in response to the environmental signal and known or not known to activate/inhibit expression of the target gene of interest, forkhead transcription factors, nuclear receptors, POU-domain proteins, SMAD, Nrf2, FOX01, NF-kB, USF2, NFAT, EGR1, STAT3, and SREBP.

5. (canceled)

6. The engineered non-naturally occurring system of claim 1, or the sgCNA subcomponent thereof, wherein the gene of interest is at least one selected from the group consisting of:
(i) a gene whose expression:
(a) produces a beneficial cellular response to the environmental signal(s) but whose expression is undetectable or is increased by the PGM relative to the gene expression level in the absence of the PGM(s); or
(b) produces a detrimental effect to the cell and its expression is decreased by the PGM in response to the environmental signal(s), relative to the gene expression level in the absence of the PGM(s); and
(ii) a gene that encodes a protein, a microRNA, or a long noncoding RNA.

7. (canceled)

8. The engineered non-naturally occurring system of claim 1, or the sgCNA subcomponent thereof, wherein the signal is at least one selected from the group consisting of a physical signal, a light signal, UV light, ionizing radiation, heat/temperature, hyperosmotic or hypoosmotic conditions, a mechanical signal, pressure, touch, movement of sound waves, blood pressure, a chemical signal, a growth factor, a cytokine, a chemokine, cyclic AMP, a hormone, a neurotransmitter, an extracellular matrix component, a bacterial antigen, a viral antigen, a lipid, a lipopolysaccharide, gas levels, oxygen levels, nitric oxide levels, ion levels, calcium levels, sodium levels, pH, a reactive oxygen species, a heavy metal, oxidized LDL, a free radical, a cell-cell signal, T-cell binding, and cell-cell contact.

9. (canceled)

10. The engineered non-naturally occurring system of claim 1, or the sgCNA subcomponent thereof, wherein the engineered non-naturally occurring system, or the sgCNA subcomponent thereof, comprises a TF-binding module, wherein the TF-binding module comprises at least one selected from the group consisting of:
(i) at least one TF-Binding segment (TFBS), wherein the TF-binding segment comprises DNA;
(ii) at least one TF-Binding segment (TFBS), wherein the TF-binding segment comprises RNA;
(iii) at least one TF-Binding segment (TFBS), wherein the TF-binding segment comprises a DNA aptamer or RNA aptamer selected for binding to the endogenous transcription factor;
(iv) a TFBS comprising a double-stranded segment of DNA containing at least one TF response element; and
(v) a sequence derived from a naturally occurring RNA.

11-12. (canceled)

13. The engineered non-naturally occurring system of claim 1, or the sgCNA subcomponent thereof, wherein the strands of the DNA portion of the sgRNA form a duplex and are joined by a loop sequence, wherein the loop sequence comprises at least one feature selected from the group consisting of:
(i) being any length;
(ii) comprising four nucleotides; and
(iii) comprising the sequence 5′-guanosine-adenosine-adenosine-adenosine-3′.

14-15. (canceled)

16. The engineered non-naturally occurring system of claim 1, or the sgCNA subcomponent thereof, wherein the crRNA comprises at least one selected from the group consisting of:
(i) at least 10, 11, 12, 13, 14, 15, 16, 17, 18, 19, 20, 21, 22, 23, 24, or 25 contiguous nucleobases that are at least 80%, 85%, 90%, 95%, 98%, 99%, or 100% complementary to the target nucleic acid sequence of the target gene of interest; and
(ii) any one of the following sequences: SEQ ID NO:6 to SEQ ID NO:34.

17-20. (canceled)

21. The engineered non-naturally occurring system of claim 1, or the sgCNA subcomponent thereof, wherein the PGM comprises crRNA, TFBS, and tracrRNA, in a molecular arrangement selected from the group consisting of:
5′-crRNA-TFBS-tracrRNA-3′;
5′-crRNA-tracrRNA′-TFBS-tracrRNA″-3′ wherein the TFBS is integrated anywhere within the sequence of the tracrRNA, including as an extension to one or more of its hairpin structures;
5′-crRNA-tracrRNA-TFBS-3′;
5′-crRNA-TFBS-tracrRNA′-TFBS-tracrRNA″-3′;
5′-crRNA-TFBS-tracrRNA-TFBS-3′;
5′-crRNA-tracrRNA′-TFBS-tracrRNA″-TFBS-3′;
5′-crRNA-TFBS-tracrRNA′-TFBS-tracrRNA″-TFBS-3′;
5′-crRNA-TFBS-tracrRNA′-TFBS-tracrRNA″-TFBS-tracrRNA′″-3′;
5′-crRNA-TFBS-tracrRNA′-TFBS-tracrRNA″-TFBS-tracrRNA′″-TFBS-tracrRNA″″-3′;
5′-crRNA-tracrRNA′-TFBS-tracrRNA″-TFBS-tracrRNA′″-TFBS-3′;
5′-crRNA-tracrRNA′-TFBS-tracrRNA″-TFBS-tracrRNA′″-TFBS-tracrRNA″″-TFBS-3′;
5′-crRNA-TFBS-tracrRNA-TFBS-tracrRNA-TFBS-tracrRNA-TFBS-3′;
5′-crRNA-TFBS-tracrRNA-TFBS-tracrRNA-TFBS-tracrRNA-TFBS-tracrRNA-TFBS-3′;
5′-crRNA-tracrRNA′-TFBS-tracrRNA″-TFBS-tracrRNA′″-3′; and
5′-crRNA-tracrRNA′-TFBS-tracrRNA″-TFBS-tracrRNA′″-TFBS-tracrRNA″″-3′;
wherein tracrRNA′, tracrRNA″, tracrRNA′″, and tracrRNA″″ are successive segments of the complete tracrRNA sequence.

22. The engineered non-naturally occurring system of claim 1, or the sgCNA subcomponent thereof, wherein the PGM comprises at least one selected from the group consisting of:
(i) one or more different TFBS, including response elements to multiple different transcription factors;
(ii) a nucleic acid backbone with one or more different TFBS wherein continuity of the nucleic acid backbone is broken at one or more positions and the full nucleic acid sequence assembles by base pairing of nucleotides from different strands;
(iii) a nucleic acid backbone with one or more different TFBS wherein continuity of the nucleic acid backbone is broken at one or more positions and the full nucleic acid sequence assembles by base pairing of nucleotides from different strands, wherein the discontinuity of the nucleic acid backbone is within one or more TFBS; and
(iv) a TFBS separated from the cRNA or tracrRNA by a linker of at least 1, 5, 10 20, or 30 DNA, RNA, or modified nucleotides.

23-25. (canceled)

26. An isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of claim 1.

27. The isolated nucleic acid of claim 26, wherein the nucleic acid comprises at least one feature selected from the group consisting of:
(i) contains one or more modified or non-natural nucleotides; and
(ii) is 5, 10, 11, 12, 13, 14, 15, 16, 17, 18, 19, 20, 21, 22, 23, 24, 25, 26, 27, 28, 29, 30, 35, 40, 45, 50, 100, 200, 300, 400, 500, 1-5, 5-10, 10-20, 20-30, 30-40, 40-50, 50-60 60-70, 70-80, 80-90, 90-100, 100-125, 125-150, 150-200, 200-300, 300-400, or 400-500 bases long.

28. (canceled)

29. A vector comprising the isolated nucleic acid of claim 26 under the control of a heterologous promoter.

30. A virus comprising the isolated nucleic acid of claim 26.

31. A cell comprising at least one selected from the group consisting of:
(i) the system of claim 1;
(ii) an isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of (i);
(iii) a vector comprising the isolated nucleic acid of (ii) under the control of a heterologous promoter;
(iv) an AAV vector comprising an isolated nucleic acid of (ii) under the control of a heterologous promoter;
(v) a virus comprising an isolated nucleic acid of (ii); and
(vi) a lentivirus or adenovirus comprising an isolated nucleic acid of (ii).

32. The cell of claim 31, wherein the cell is selected from the group consisting of a mammalian cell, a cell of a non-human primate, and a human cell.

33. A composition comprising at least one selected from the group consisting of:
(i) the system of claim 1;
(ii) an isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of (i);
(iii) a vector comprising the isolated nucleic acid of (ii) under the control of a heterologous promoter;
(iv) an AAV vector comprising an isolated nucleic acid of (ii) under the control of a heterologous promoter;
(v) a virus comprising an isolated nucleic acid of (ii);
(vi) a lentivirus or adenovirus comprising an isolated nucleic acid of (ii),
(vii) a cell comprising (i), (ii), (iii), (iv), (v), or (vi); and
(viii) a cationic or ionizable lipid, cationic or ionizable polymer, or nanoparticle.

34. (canceled)

35. The composition of claim 33, wherein the composition is a pharmaceutical composition further comprising a pharmaceutically acceptable excipient.

36. A method for reversibly modifying expression of a target gene of interest in a cell in response to one or more intracellular or extracellular environmental signal(s), comprising contacting the cell with at least one selected from the group consisting of:
(i) the system of claim 1;
(ii) an isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of (i);
(iii) a vector comprising the isolated nucleic acid of (ii) under the control of a heterologous promoter;
(iv) an AAV vector comprising an isolated nucleic acid of (ii) under the control of a heterologous promoter;
(v) a virus comprising the isolated nucleic acid of (ii);
(vi) a lentivirus or adenovirus comprising the isolated nucleic acid of (ii);
(vii) a cell comprising (i), (ii), (iii), (iv), (v), or (vi); and
(viii) a composition comprising (i), (ii), (iii), (iv), (v), (vi), or (vii).

37. The method of claim 36, wherein the cell is selected from the group consisting of: a mammalian cell, a cell of a non-human primate, and a human cell.

38. A method of treating a disease, disorder, or injury in a subject in need thereof, the method comprising administering to the subject a therapeutically effective amount of at least one selected from the group consisting of:
(i) the system of claim 1;
(ii) an isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of (i);
(iii) a vector comprising the isolated nucleic acid of (ii) under the control of a heterologous promoter;
(iv) an AAV vector comprising an isolated nucleic acid of (ii) under the control of a heterologous promoter;
(v) a virus comprising the isolated nucleic acid of (ii);
(vi) a lentivirus or adenovirus comprising the isolated nucleic acid of (ii);
(vii) a cell comprising (i), (ii), (iii), (iv), (v), or (vi); and
(viii) a composition comprising (i), (ii), (iii), (iv), (v), (vi), or (vii).

39. The method of claim 38, wherein the disease, disorder, or injury is selected from the group consisting of cellular stress, an excisional or incisional wound, radiation exposure, viral or bacterial infection, sepsis, diabetic nephropathy, atherosclerosis, cystic fibrosis, Alzheimer's disease, oxidative stress, ischemia-reperfusion injury, inflammation, cancer, anti-cancer agent resistance, a genetic disease, a proliferative disease or disorder, inflammatory disease or disorder, autoimmune disease or disorder, liver disease or disorder, spleen disease or disorder, lung disease or disorder, hematological disease or disorder, neurological disease or disorder, gastrointestinal (GI) tract disease or disorder, genitourinary disease or disorder, infectious disease or disorder, musculoskeletal disease or disorder, endocrine disease or disorder, metabolic disease or disorder, immune disease or disorder, central nervous system (CNS) disease or disorder, neurological disease or disorder, ophthalmic disease or disorder, and a cardiovascular disease or disorder.

40. (canceled)

41. A kit comprising at least one selected from the group consisting of:
(i) the system of claim 1;
(ii) an isolated nucleic acid comprising any one or more of the sgCNA, crRNA, tracrRNA, transcription factor binding site, any other segment of the sgCNA of the PGM, or a sequence encoding the DNA binding polypeptide of the PGM, of the system of (i);
(iii) a vector comprising the isolated nucleic acid of (ii) under the control of a heterologous promoter;
(iv) an AAV vector comprising the isolated nucleic acid of (ii) under the control of a heterologous promoter;
(v) a virus comprising the isolated nucleic acid of (ii);
(vi) a lentivirus or adenovirus comprising the isolated nucleic acid of (ii);
(vii) a cell comprising (i), (ii), (iii), (iv), (v), or (vi);
(viii) a composition comprising (i), (ii), (iii), (iv), (v), (vi), or (vii);
(ix) a container; and
(x) instructions for using the kit.

42. The vector of claim 29, wherein the vector is an AAV vector.

43. The virus of claim 30, wherein the virus is a lentivirus or adenovirus.


SEQUENCE LISTING (76 sequences)

SEQ ID NO: 1 - DNA, 33 bp, Homo sapiens
  source 1..33: mol_type = genomic DNA
  misc_feature 11..23: note = This region may be absent
rrrcwwgyyynnnnnnnnnnnnnrrrcwwgyyy

SEQ ID NO: 2 - DNA, 13 bp, Homo sapiens
  source 1..13: mol_type = genomic DNA
ggtcannntgacc

SEQ ID NO: 3 - DNA, 10 bp, Homo sapiens
  source 1..10: mol_type = genomic DNA
kgggcggrry

SEQ ID NO: 4 - DNA, 167 bp, synthetic construct
  source 1..167: mol_type = other DNA
  misc_feature 1..32: note = RNA
  misc_feature 33..102: note = DNA
  misc_feature 103..167: note = RNA
cggaggcagtcccggctcgcgttttagagctaaccctgacttgacgtatgactcagcacaatggcgaaagccattgtgctgagtcatacgtcaagtcagggttagcaagttaaaataaggctagtccgttatcaacttgaaaaagtggcacccgagtcggtgctttt

SEQ ID NO: 5 - DNA, 11 bp, Homo sapiens
  source 1..11: mol_type = genomic DNA
atgactcagca

SEQ ID NO: 6 - RNA, 20 nt, synthetic construct
  source 1..20: mol_type = other RNA
cgtccacgaaacgtcctgca

SEQ ID NO: 7 - RNA, 20 nt, synthetic construct
  source 1..20: mol_type = other RNA
gcaataattacctgcgagcc

SEQ ID NO: 8 - RNA, 20 nt, synthetic construct
  source 1..20: mol_type = other RNA
gagccgtgcaggacgtttcg

SEQ ID NO: 9 - RNA, 20 nt, synthetic construct
  source 1..20: mol_type = other RNA
caggcgtcgcccgcggacgt

SEQ ID NO: 10 - RNA, 20 nt, synthetic construct
  source 1..20: mol_type = other RNA
ttattgccagcggagcccgc

SEQ ID NO: 11 - RNA, 20 nt, synthetic construct
  source 1..20: mol_type = other RNA
gtgcctttctccgacgtccg

SEQ ID NO: 12 - RNA, 20 nt, synthetic construct
  source 1..20: mol_type = other RNA
cggaggcagtcccggctcgc

SEQ ID NO: 13 - RNA, 20 nt, synthetic construct
  source 1..20: mol_type = other RNA
tcccgggcacccctcgccct

SEQ ID NO: 14 - RNA, 20 nt, synthetic construct
  source 1..20: mol_type = other RNA
ctgcctccgccctggcactg

SEQ ID NO: 15 - RNA, 20 nt, synthetic construct
  source 1..20: mol_type = other RNA
gagggcgaggggtgcccggg

SEQ ID NO: 16 - RNA, 20 nt, synthetic construct
  source 1..20: mol_type = other RNA
acatcggaagaactgaaccc

SEQ ID NO: 17 - RNA, 20 nt, synthetic construct
  source 1..20: mol_type = other RNA
ctaggtcaagagctcactgg

SEQ ID NO: 18 - RNA, 20 nt, synthetic construct
  source 1..20: mol_type = other RNA
ggaatctgagctatcaggac

SEQ ID NO: 19 - RNA, 20 nt, synthetic construct
  source 1..20: mol_type = other RNA
agaactgaacccaggactct

SEQ ID NO: 20 - RNA, 20 nt, synthetic construct
  source 1..20: mol_type = other RNA
gctctaggtcaagagctcac

SEQ ID NO: 21 - RNA, 20 nt, synthetic construct
  source 1..20: mol_type = other RNA
tctaggtcaagagctcactg

SEQ ID NO: 22 - RNA, 20 nt, synthetic construct
  source 1..20: mol_type = other RNA
gttgctactaaacaacacat

SEQ ID NO: 23 - RNA, 20 nt, synthetic construct
  source 1..20: mol_type = other RNA
tgtgttgtttagtagcaact

SEQ ID NO: 24 - RNA, 20 nt, synthetic construct
  source 1..20: mol_type = other RNA
gcaactgggttcttctgttg

SEQ ID NO: 25 - RNA, 20 nt, synthetic construct
  source 1..20: mol_type = other RNA
ttcttctgttggggaagagg

SEQ ID NO: 26 - RNA, 20 nt, synthetic construct
  source 1..20: mol_type = other RNA
gcgagtcctctcgttcgacg

SEQ ID NO: 27 - RNA, 20 nt, synthetic construct
  source 1..20: mol_type = other RNA
cagctaccacgtcgaacgag

SEQ ID NO: 28 - RNA, 20 nt, synthetic construct
  source 1..20: mol_type = other RNA
gcggactgacagctggcgag

SEQ ID NO: 29 - RNA, 20 nt, synthetic construct
  source 1..20: mol_type = other RNA
gaatcactcctgccacacgt

SEQ ID NO: 30 - RNA, 20 nt, synthetic construct
  source 1..20: mol_type = other RNA
cggactgacagctggcgaga

SEQ ID NO: 31 - RNA, 20 nt, synthetic construct
  source 1..20: mol_type = other RNA
ggcaggagtgattccaagag

SEQ ID NO: 32 - RNA, 20 nt, synthetic construct
  source 1..20: mol_type = other RNA
cgggtgatccagatgcgctg

SEQ ID NO: 33 - RNA, 20 nt, synthetic construct
  source 1..20: mol_type = other RNA
cggattaagccttctccgcc

SEQ ID NO: 34 - RNA, 20 nt, synthetic construct
  source 1..20: mol_type = other RNA
gagcccgcccacgcgagatg

SEQ ID NO: 35 - DNA, 20 bp, Homo sapiens
  source 1..20: mol_type = genomic DNA
tgcaggacgtttcgtggacg

SEQ ID NO: 36 - DNA, 20 bp, Homo sapiens
  source 1..20: mol_type = genomic DNA
ggctcgcaggtaattattgc

SEQ ID NO: 37 - DNA, 20 bp, Homo sapiens
  source 1..20: mol_type = genomic DNA
cgaaacgtcctgcacggctc

SEQ ID NO: 38 - DNA, 19 bp, Homo sapiens
  source 1..19: mol_type = genomic DNA
acgtccgcgggcgacgccg

SEQ ID NO: 39 - DNA, 20 bp, Homo sapiens
  source 1..20: mol_type = genomic DNA
gcgggctccgctggcaataa

SEQ ID NO: 40 - DNA, 20 bp, Homo sapiens
  source 1..20: mol_type = genomic DNA
cggacgtcggagaaaggcac

SEQ ID NO: 41 - DNA, 20 bp, Homo sapiens
  source 1..20: mol_type = genomic DNA
gcgagccgggactgcctccg

SEQ ID NO: 42 - DNA, 19 bp, Homo sapiens
  source 1..19: mol_type = genomic DNA
agggcgagggtgcccggga

SEQ ID NO: 43 - DNA, 19 bp, Homo sapiens
  source 1..19: mol_type = genomic DNA
cagtgccagggcgaggcag

SEQ ID NO: 44 - DNA, 20 bp, Homo sapiens
  source 1..20: mol_type = genomic DNA
cccgggcacccctcgccctc

SEQ ID NO: 45 - DNA, 20 bp, Homo sapiens
  source 1..20: mol_type = genomic DNA
gggttcagttcttccgatgt

SEQ ID NO: 46 - DNA, 20 bp, Homo sapiens
  source 1..20: mol_type = genomic DNA
ccagtgagctcttgacctag

SEQ ID NO: 47 - DNA, 20 bp, Homo sapiens
  source 1..20: mol_type = genomic DNA
gtcctgatagctcagattcc

SEQ ID NO: 48 - DNA, 20 bp, Homo sapiens
  source 1..20: mol_type = genomic DNA
agagtcctgggttcagttca

SEQ ID NO: 49 - DNA, 20 bp, Homo sapiens
  source 1..20: mol_type = genomic DNA
gtgagctcttgacctagagc

SEQ ID NO: 50 - DNA, 20 bp, Homo sapiens
  source 1..20: mol_type = genomic DNA
cagtgagctcttgacctaga

SEQ ID NO: 51 - DNA, 20 bp, Homo sapiens
  source 1..20: mol_type = genomic DNA
atgtgttgtttagtagcaac

SEQ ID NO: 52 - DNA, 20 bp, Homo sapiens
  source 1..20: mol_type = genomic DNA
agttgctactaaacaacaca

SEQ ID NO: 53 - DNA, 20 bp, Homo sapiens
  source 1..20: mol_type = genomic DNA
caacagaagaacccagttgc

SEQ ID NO: 54 - DNA, 20 bp, Homo sapiens
  source 1..20: mol_type = genomic DNA
cctcttccccaacagaagaa

SEQ ID NO: 55 - DNA, 20 bp, Homo sapiens
  source 1..20: mol_type = genomic DNA
cgtcgaacgagaggactcgc

SEQ ID NO: 56 - DNA, 20 bp, Homo sapiens
  source 1..20: mol_type = genomic DNA
ctcgttcgacgtggtagctg

SEQ ID NO: 57 - DNA, 20 bp, Homo sapiens
  source 1..20: mol_type = genomic DNA
ctcgccagctgtcagtccgc

SEQ ID NO: 58 - DNA, 20 bp, Homo sapiens
  source 1..20: mol_type = genomic DNA
acgtgtggcaggagtgattc

SEQ ID NO: 59 - DNA, 20 bp, Homo sapiens
  source 1..20: mol_type = genomic DNA
tctcgccagctgtcagtccg

SEQ ID NO: 60 - DNA, 20 bp, Homo sapiens
  source 1..20: mol_type = genomic DNA
ctcttggaatcactcctgcc

SEQ ID NO: 61 - DNA, 20 bp, Homo sapiens
  source 1..20: mol_type = genomic DNA
cagcgcatctggatcacccg

SEQ ID NO: 62 - DNA, 20 bp, Homo sapiens
  source 1..20: mol_type = genomic DNA
ggcggagaaggcttaatccg

SEQ ID NO: 63 - DNA, 20 bp, Homo sapiens
  source 1..20: mol_type = genomic DNA
catctcgcgtgggcgggctc

SEQ ID NO: 64 - DNA, 47 bp, synthetic construct
  source 1..47: mol_type = other DNA
  misc_feature 1..32: note = RNA
  misc_feature 33..47: note = DNA
cggaggcagtcccggctcgcgttttagagctaaccctgacttgacgt

SEQ ID NO: 65 - DNA, 74 bp, synthetic construct
  source 1..74: mol_type = other DNA
  misc_feature 1..10: note = DNA
  misc_feature 11..74: note = RNA
aagtcagggttagcaagttaaaataaggctagtccgttatcaacttgaaaaagtggcaccgagtcggtgctttt

SEQ ID NO: 66 - DNA, 45 bp, synthetic construct
  source 1..45: mol_type = other DNA
atgactcagcacaatggcgaaagccattgtgctgagtcatacgtc

SEQ ID NO: 67 - RNA, 20 nt, synthetic construct
  source 1..20: mol_type = other RNA
cggaggcagtcccggctcgc

SEQ ID NO: 68 - DNA, 11 bp, synthetic construct
  source 1..11: mol_type = other DNA
atgactcagca

SEQ ID NO: 69 - DNA, 166 bp, synthetic construct
  source 1..166: mol_type = other DNA
  misc_feature 1..32: note = RNA
  misc_feature 33..102: note = DNA
  misc_feature 103..166: note = DNA
cggaggcagtcccggctcgcgttttagagctaaccctgacttgacgtatgactcagcacaatggcgaaagccattgtgctgagtcatacgtcaagtcagggttagcaagttaaaataaggctagtccgttatcaacttgaaaaagtggcaccgagtcggtgctttt

SEQ ID NO: 70 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 71 - DNA, 90 bp, synthetic construct
  source 1..90: mol_type = other DNA
cctcggcgcccctgcccccgcccccagtgccagggcggaggcagtcccggctcgcaggtaattattgccagcggagcccgccggggagcg

SEQ ID NO: 72 - DNA, 90 bp, synthetic construct
  source 1..90: mol_type = other DNA
cgctccccggcgggctccgctggcaataattacctgcgagccgggactgcctccgccctggcactgggggcgggggcaggggcgccgagg

SEQ ID NO: 73 - DNA, 90 bp, synthetic construct
  source 1..90: mol_type = other DNA
cctcggcgcccctgcccccgcccccagtgccagggggacgcgcgggcaccgcttcaggtaattattgccagcggagcccgccggggagcg

SEQ ID NO: 74 - DNA, 90 bp, synthetic construct
  source 1..90: mol_type = other DNA
cgctccccggcgggctccgctggcaataattacctgaagcggtgcccgcgcgtccccctggcactgggggcgggggcaggggcgccgagg

SEQ ID NO: 75 - DNA, 15 bp, synthetic construct
  source 1..15: mol_type = other DNA
accctgacttgacgt

SEQ ID NO: 76 - DNA, 10 bp, synthetic construct
  source 1..10: mol_type = other DNA
aagtcagggt